SEQUENCE LISTING (141 sequences)

SEQ ID NO: 1 - protein, 279 aa, synthetic construct
DPIVMIDNDKPITGGGGSGSQVQLQESGPGLVKPSETLSLTCTVSGYSISSGYYWGWIRQPPGKGLEWIGSIYHSGSTYYNPSLKTRVTISVDTSKNQFSLKLSSVTAADTAVYYCARAVVGIVVVPAAGRRAFDIWGQGTMVTVSSGGGGSGGGGSGGGGSGGGGSGGGGSQSALTQPPSASGSPGQSVTISCTGTSSDVGGYNYVSWYQQHPGKAPKLMIYEVSKRPSGVPDRFSGSKSGNTASLTVSGLQAEDEADYYCSSYAGSNNLVFGGGTKV

SEQ ID NO: 2 - protein, 269 aa, synthetic construct
AHIVMVDAYKPTKGGGGSGSEVQLVESGGGLVQPGGSLRLSCAASGITVSSNYMNWVRQAPGKGLEWVSLIYSGGSTYYADSVKGRFTISRDNSKNTLYLQMNSLRAEDTAVYHCARDLVVYGMDVWGQGTTVTVSSGGGGSGGGGSGGGGSGGGGSGGGGSEIVLTQSPGTLSLSPGERATLSCRASQSVSSSYLAWYQQKPGQAPRLLIYGASSRATGIPDRFSGSGSGTDFTLTISRLEPEDFAVYYCQQYGSSPTFGQGTKLEIK

SEQ ID NO: 3 - protein, 270 aa, synthetic construct
KLGDIEFIKVNKGGGGSGSQVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCARDFSHQQLVPSWGQGTLVTVSSGGGGSGGGGSGGGGSGGGGSGGGGSDIQLTQSPSFLSASVGDRVTITCRASQGISSYLAWYQQKPGKAPKLLIYAASTLQSGVPSRFSGSGSGTEFTLTISSLQPEDFATYYCQQLNSYPLTFGGGTKVEIK

SEQ ID NO: 4 - protein, 6 aa, synthetic construct
HHHHHH

SEQ ID NO: 5 - protein, 227 aa, synthetic construct
SPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKGIEENLYFQS

SEQ ID NO: 6 - protein, 159 aa, synthetic construct
VSGNLKVEFYNSNPSDTTNSINPQFKVTNTGSSAIDLSKLTLRYYYTVDGQKDQTFWCDHAAIIGSNGSYNGITSNVKGTFVKMSSSTNNADTYLEISFTGGTLEPGAHVQIQGRFAKNDWSNYTQSNDYSFKSASQFVEWDQVTAYLNGVLVWGKESR

SEQ ID NO: 7 - protein, 695 aa, synthetic construct
MNHKVHHHHHHIEGRHMSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKGIEENLYFQSHMELGTLEGEFDPIVMIDNDKPITGGGGSGSQVQLQESGPGLVKPSETLSLTCTVSGYSISSGYYWGWIRQPPGKGLEWIGSIYHSGSTYYNPSLKTRVTISVDTSKNQFSLKLSSVTAADTAVYYCARAVVGIVVVPAAGRRAFDIWGQGTMVTVSSGGGGSGGGGSGGGGSGGGGSGGGGSQSALTQPPSASGSPGQSVTISCTGTSSDVGGYNYVSWYQQHPGKAPKLMIYEVSKRPSGVPDRFSGSKSGNTASLTVSGLQAEDEADYYCSSYAGSNNLVFGGGTKVDSVSGNLKVEFYNSNPSDTTNSINPQFKVTNTGSSAIDLSKLTLRYYYTVDGQKDQTFWCDHAAIIGSNGSYNGITSNVKGTFVKMSSSTNNADTYLEISFTGGTLEPGAHVQIQGRFAKNDWSNYTQSNDYSFKSASQFVEWDQVTAYLNGVLVWGKESR

SEQ ID NO: 8 - protein, 685 aa, synthetic construct
MNHKVHHHHHHIEGRHMSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKGIEENLYFQSHMELGTLEGEFAHIVMVDAYKPTKGGGGSGSEVQLVESGGGLVQPGGSLRLSCAASGITVSSNYMNWVRQAPGKGLEWVSLIYSGGSTYYADSVKGRFTISRDNSKNTLYLQMNSLRAEDTAVYHCARDLVVYGMDVWGQGTTVTVSSGGGGSGGGGSGGGGSGGGGSGGGGSEIVLTQSPGTLSLSPGERATLSCRASQSVSSSYLAWYQQKPGQAPRLLIYGASSRATGIPDRFSGSGSGTDFTLTISRLEPEDFAVYYCQQYGSSPTFGQGTKLEIKDSVSGNLKVEFYNSNPSDTTNSINPQFKVTNTGSSAIDLSKLTLRYYYTVDGQKDQTFWCDHAAIIGSNGSYNGITSNVKGTFVKMSSSTNNADTYLEISFTGGTLEPGAHVQIQGRFAKNDWSNYTQSNDYSFKSASQFVEWDQVTAYLNGVLVWGKESR

SEQ ID NO: 9 - protein, 686 aa, synthetic construct
MNHKVHHHHHHIEGRHMSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKGIEENLYFQSHMELGTLEGEFKLGDIEFIKVNKGGGGSGSQVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCARDFSHQQLVPSWGQGTLVTVSSGGGGSGGGGSGGGGSGGGGSGGGGSDIQLTQSPSFLSASVGDRVTITCRASQGISSYLAWYQQKPGKAPKLLIYAASTLQSGVPSRFSGSGSGTEFTLTISSLQPEDFATYYCQQLNSYPLTFGGGTKVEIKDSVSGNLKVEFYNSNPSDTTNSINPQFKVTNTGSSAIDLSKLTLRYYYTVDGQKDQTFWCDHAAIIGSNGSYNGITSNVKGTFVKMSSSTNNADTYLEISFTGGTLEPGAHVQIQGRFAKNDWSNYTQSNDYSFKSASQFVEWDQVTAYLNGVLVWGKESR

SEQ ID NO: 10 - protein, 25 aa, synthetic construct
QVQLQESGPGLVKPSETLSLTCTVS

SEQ ID NO: 11 - protein, 9 aa, synthetic construct
GYSISSGYY

SEQ ID NO: 12 - protein, 17 aa, synthetic construct
WGWIRQPPGKGLEWIGS

SEQ ID NO: 13 - protein, 7 aa, synthetic construct
IYHSGST

SEQ ID NO: 14 - protein, 38 aa, synthetic construct
YYNPSLKTRVTISVDTSKNQFSLKLSSVTAADTAVYYC

SEQ ID NO: 15 - protein, 21 aa, synthetic construct
ARAVVGIVVVPAAGRRAFDIW

SEQ ID NO: 16 - protein, 25 aa, synthetic construct
QSALTQPPSASGSPGQSVTISCTGT

SEQ ID NO: 17 - protein, 9 aa, synthetic construct
SSDVGGYNY

SEQ ID NO: 18 - protein, 17 aa, synthetic construct
VSWYQQHPGKAPKLMIY

SEQ ID NO: 19 - skipped

SEQ ID NO: 20 - protein, 36 aa, synthetic construct
KRPSGVPDRFSGSKSGNTASLTVSGLQAEDEADYYC

SEQ ID NO: 21 - protein, 18 aa, synthetic construct
SSYAGSNNLVFGGGTKVD

SEQ ID NO: 22 - protein, 25 aa, synthetic construct [sequence omitted]

SEQ ID NO: 23 - protein, 8 aa, synthetic construct
GITVSSNY

SEQ ID NO: 24 - protein, 17 aa, synthetic construct
MNWVRQAPGKGLEWVSL

SEQ ID NO: 25 - protein, 8 aa, synthetic construct
IYSGGSTY

SEQ ID NO: 26 - protein, 37 aa, synthetic construct
YADSVKGRFTISRDNSKNTLYLQMNSLRAEDTAVYHC

SEQ ID NO: 27 - protein, 20 aa, synthetic construct
ARDLVVYGMDVWGQGTTVTV

SEQ ID NO: 28 - protein, 23 aa, synthetic construct
EIVLTQSPGTLSLSPGERATLSC

SEQ ID NO: 29 - protein, 10 aa, synthetic construct
RASQSVSSSY

SEQ ID NO: 30 - protein, 17 aa, synthetic construct
LAWYQQKPGQAPRLLIY

SEQ ID NO: 31 - skipped

SEQ ID NO: 32 - protein, 56 aa, synthetic construct
SRATGIPDRFSGSGSGTDFTLTISRLEPEDFAVYYCQQYGSSPTFGQGTKLEIKVD

SEQ ID NO: 33 - protein, 20 aa, synthetic construct
QQYGSSPTFGQGTKLEIKVD

SEQ ID NO: 34 - protein, 25 aa, synthetic construct
QVQLVESGGGLVKPGGSLRLSCAAS

SEQ ID NO: 35 - protein, 8 aa, synthetic construct
GFTFSDYY

SEQ ID NO: 36 - protein, 17 aa, synthetic construct
MSWIRQAPGKGLEWVSY

SEQ ID NO: 37 - protein, 8 aa, synthetic construct
ISSSGSTI

SEQ ID NO: 38 - protein, 38 aa, synthetic construct
YYADSVKGRFTISRDNAKNSLYLQMNSLRAEDTAVYYC

SEQ ID NO: 39 - protein, 21 aa, synthetic construct
ARDFSHQQLVPSWGQGTLVTV

SEQ ID NO: 40 - protein, 26 aa, synthetic construct
DIQLTQSPSFLSASVGDRVTITCRAS

SEQ ID NO: 41 - protein, 6 aa, synthetic construct
QGISSY

SEQ ID NO: 42 - protein, 17 aa, synthetic construct
LAWYQQKPGKAPKLLIY

SEQ ID NO: 43 - skipped

SEQ ID NO: 44 - protein, 36 aa, synthetic construct
TLQSGVPSRFSGSGSGTEFTLTISSLQPEDFATYYC

SEQ ID NO: 45 - protein, 21 aa, synthetic construct
QQLNSYPLTFGGGTKVEIKVD

SEQ ID NO: 46 - protein, 227 aa, synthetic construct
AHIVMVDAYKPTKGGGGSGSQVQLKESGPGLVAPSQSLSITCTVSGFSLTRYGVHWVRQPPGKGLEWLGVIWADGSTYYNSALMSRLSISKDNSKSQVFLNMNSLQTDDTAKYYCGGGGSGGGGSGGGGSGGGGSGGGGSQIVLTQSPAIMSASPGEKVTMTCSASSTVSFIYWYQQKPGSSPRLLIYDTSNPASGVPVRFSGSGCGTSYYLTISRMEAEDAATYYC

SEQ ID NO: 47 - protein, 232 aa, synthetic construct
KLGDIEFIKVNKGGGGSGSQITLKESGPTLVKPTQTLTLTCSFSGFSLSTSGVGVGWIRQPPGKALEWLALIDWDDNKYHTTSLKTRLTISKDTSKNQVVLTMTNMDPVDTATYYCGGGGSGGGGSGGGGSGGGGSGGGGSELVLTQPPSVSAAPGQKVTISCSGSSSNIGNNYVSWYQQLPGTAPKLLIYDNNKRPSGIPDRFSGSKSGTSATLGACITGLQTGDEADYYC

SEQ ID NO: 48 - protein, 232 aa, synthetic construct
DPIVMIDNDKPITGGGGSGSQITLKESGPTLVKPTQTLTLTCTFSGFSLSISGVGVGWLRQPPGKALEWLALIYWDDDKRYSPSLKSRLTISKDTSKNQVVLKMTNIDPVDTATYYCGGGGSGGGGSGGGGSGGGGSGGGGSQSALTQPASVSGSPGQSITISCTATSSDVGDYNYVSWYQQHPGKAPKLMIFEVSDRPSGISNRFSGSKSGNTASLTISGLQAEDEADYYC

SEQ ID NO: 49 - protein, 643 aa, synthetic construct
MNHKVHHHHHHIEGRHMSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKGIEENLYFQSHMELGTLEGEFAHIVMVDAYKPTKGGGGSGSQVQLKESGPGLVAPSQSLSITCTVSGFSLTRYGVHWVRQPPGKGLEWLGVIWADGSTYYNSALMSRLSISKDNSKSQVFLNMNSLQTDDTAKYYCGGGGSGGGGSGGGGSGGGGSGGGGSQIVLTQSPAIMSASPGEKVTMTCSASSTVSFIYWYQQKPGSSPRLLIYDTSNPASGVPVRFSGSGCGTSYYLTISRMEAEDAATYYCVDVSGNLKVEFYNSNPSDTTNSINPQFKVTNTGSSAIDLSKLTLRYYYTVDGQKDQTFWCDHAAIIGSNGSYNGITSNVKGTFVKMSSSTNNADTYLEISFTGGTLEPGAHVQIQGRFAKNDWSNYTQSNDYSFKSASQFVEWDQVTAYLNGVLVWGKESR

SEQ ID NO: 50 - protein, 648 aa, synthetic construct
MNHKVHHHHHHIEGRHMSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKGIEENLYFQSHMELGTLEGEFKLGDIEFIKVNKGGGGSGSQITLKESGPTLVKPTQTLTLTCSFSGFSLSTSGVGVGWIRQPPGKALEWLALIDWDDNKYHTTSLKTRLTISKDTSKNQVVLTMTNMDPVDTATYYCGGGGSGGGGSGGGGSGGGGSGGGGSELVLTQPPSVSAAPGQKVTISCSGSSSNIGNNYVSWYQQLPGTAPKLLIYDNNKRPSGIPDRFSGSKSGTSATLGACITGLQTGDEADYYCVDVSGNLKVEFYNSNPSDTTNSINPQFKVTNTGSSAIDLSKLTLRYYYTVDGQKDQTFWCDHAAIIGSNGSYNGITSNVKGTFVKMSSSTNNADTYLEISFTGGTLEPGAHVQIQGRFAKNDWSNYTQSNDYSFKSASQFVEWDQVTAYLNGVLVWGKESR

SEQ ID NO: 51 - protein, 648 aa, synthetic construct
MNHKVHHHHHHIEGRHMSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKGIEENLYFQSHMELGTLEGEFDPIVMIDNDKPITGGGGSGSQITLKESGPTLVKPTQTLTLTCTFSGFSLSISGVGVGWLRQPPGKALEWLALIYWDDDKRYSPSLKSRLTISKDTSKNQVVLKMTNIDPVDTATYYCGGGGSGGGGSGGGGSGGGGSGGGGSQSALTQPASVSGSPGQSITISCTATSSDVGDYNYVSWYQQHPGKAPKLMIFEVSDRPSGISNRFSGSKSGNTASLTISGLQAEDEADYYCVDVSGNLKVEFYNSNPSDTTNSINPQFKVTNTGSSAIDLSKLTLRYYYTVDGQKDQTFWCDHAAIIGSNGSYNGITSNVKGTFVKMSSSTNNADTYLEISFTGGTLEPGAHVQIQGRFAKNDWSNYTQSNDYSFKSASQFVEWDQVTAYLNGVLVWGKESR

SEQ ID NO: 52 - protein, 31 aa, synthetic construct
QVQLKESGPGLVAPSQSLSITCTVSGFSLTR

SEQ ID NO: 53 - protein, 8 aa, synthetic construct
GFSLTRYG

SEQ ID NO: 54 - protein, 17 aa, synthetic construct
VHWVRQPPGKGLEWLGV

SEQ ID NO: 55 - protein, 7 aa, synthetic construct
IWADGST

SEQ ID NO: 56 - protein, 38 aa, synthetic construct
YYNSALMSRLSISKDNSKSQVFLNMNSLQTDDTAKYYC

SEQ ID NO: 57 - protein, 26 aa, synthetic construct
QIVLTQSPAIMSASPGEKVTMTCSAS

SEQ ID NO: 58 - protein, 5 aa, synthetic construct
STVSF

SEQ ID NO: 59 - protein, 17 aa, synthetic construct
IYWYQQKPGSSPRLLIY

SEQ ID NO: 60 - skipped

SEQ ID NO: 61 - protein, 36 aa, synthetic construct
NPASGVPVRFSGSGCGTSYYLTISRMEAEDAATYYC

SEQ ID NO: 62 - protein, 25 aa, synthetic construct
QITLKESGPTLVKPTQTLTLTCSFS

SEQ ID NO: 63 - protein, 10 aa, synthetic construct
GFSLSTSGVG

SEQ ID NO: 64 - protein, 17 aa, synthetic construct
VGWIRQPPGKALEWLAL

SEQ ID NO: 65 - protein, 7 aa, synthetic construct
IDWDDNK

SEQ ID NO: 66 - protein, 38 aa, synthetic construct
YHTTSLKTRLTISKDTSKNQVVLTMTNMDPVDTATYYC

SEQ ID NO: 67 - protein, 25 aa, synthetic construct
ELVLTQPPSVSAAPGQKVTISCSGS

SEQ ID NO: 68 - protein, 8 aa, synthetic construct
SSNIGNNY

SEQ ID NO: 69 - protein, 17 aa, synthetic construct
VSWYQQLPGTAPKLLIY

SEQ ID NO: 70 - skipped

SEQ ID NO: 71 - protein, 38 aa, synthetic construct
KRPSGIPDRFSGSKSGTSATLGACITGLQTGDEADYYC

SEQ ID NO: 72 - protein, 25 aa, synthetic construct
QITLKESGPTLVKPTQTLTLTCTFS

SEQ ID NO: 73 - protein, 10 aa, synthetic construct
GFSLSISGVG

SEQ ID NO: 74 - protein, 17 aa, synthetic construct
VGWLRQPPGKALEWLAL

SEQ ID NO: 75 - protein, 7 aa, synthetic construct
IYWDDDK

SEQ ID NO: 76 - protein, 38 aa, synthetic construct
RYSPSLKSRLTISKDTSKNQVVLKMTNIDPVDTATYYC

SEQ ID NO: 77 - protein, 25 aa, synthetic construct
QSALTQPASVSGSPGQSITISCTAT

SEQ ID NO: 78 - protein, 9 aa, synthetic construct
SSDVGDYNY

SEQ ID NO: 79 - protein, 17 aa, synthetic construct
VSWYQQHPGKAPKLMIF

SEQ ID NO: 80 - skipped

SEQ ID NO: 81 - protein, 36 aa, synthetic construct
DRPSGISNRFSGSKSGNTASLTISGLQAEDEADYYC

SEQ ID NO: 82 - protein, 287 aa, synthetic construct
AHIVMVDAYKPTKGGGGSGSQVQLVQSGAEVKKPGSSVKVSCKASGGIFNTYTISWVRQAPGQGLEWMGRIILMSGMANYAQKIQGRVTITADKSTSTAYMELTSLRSDDTAVYYCARGFNGNYYGWGDDDAFDIWGQGTLVTVYSGGGGSGGGGSGGGGSGGGGSGGGGSQTVLTQPPSVSGAPGQRVTISCTGSNSNIGAGYDVHWYQQLPGTAPKLLICGNSNRPSGVPDRFSGSKSGTSASLAITGLQAEDEADYYCQSYDSSLSGPNWVFGGGTKLTVLGQP

SEQ ID NO: 83 - protein, 247 aa, synthetic construct
KLGDIEFIKVNKGGGGSGSQVRLVQSGAEVKKSGESLKISCKGSGYSFTSYWIGWVRQMPGKGLEWMGIIYPGDSDTRYSPSFQGQVTISADKSISTVYLQWSSLKASDTAMYYCARQWSHYTYDYYYGGGGSGGGGSGGGGSGGGGSGGGGSQSVLTQPASVSGSPGQSITISCTGISSDVGGYNSVSWYQQHPGKAPKLMIYDVTNRPSGVSNRFSGSKSGNTASLTISGLQAEDEADYYCSSYT

SEQ ID NO: 84 - protein, 255 aa, synthetic construct
DPIVMIDNDKPITGGGGSGSQVQLVESGGVVVQPGGSLRLSCAASGFTFHDHTMHWVRQAPGKGLEWVSLITWNGGTIHYSDSVKGRFTISRDNSKNSLYLQMNSLRTEDTALYYCAKDLGRGGWYLPSDAGGGGSGGGGSGGGGSGGGGSGGGGSQSVLTQPPSASGTPGQRVTISCSGSSANIGSNTVNWYQHLPGTAPKLLIYSNNQRPSGVPDRFSGSKSGTSASLAISGLQSEDEADYYCAAWDDSLKGV

SEQ ID NO: 85 - protein, 703 aa, synthetic construct
MNHKVHHHHHHIEGRHMSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKGIEENLYFQSHMELGTLEGEFAHIVMVDAYKPTKGGGGSGSQVQLVQSGAEVKKPGSSVKVSCKASGGIFNTYTISWVRQAPGQGLEWMGRIILMSGMANYAQKIQGRVTITADKSTSTAYMELTSLRSDDTAVYYCARGFNGNYYGWGDDDAFDIWGQGTLVTVYSGGGGSGGGGSGGGGSGGGGSGGGGSQTVLTQPPSVSGAPGQRVTISCTGSNSNIGAGYDVHWYQQLPGTAPKLLICGNSNRPSGVPDRFSGSKSGTSASLAITGLQAEDEADYYCQSYDSSLSGPNWVFGGGTKLTVLGQPVDVSGNLKVEFYNSNPSDTTNSINPQFKVTNTGSSAIDLSKLTLRYYYTVDGQKDQTFWCDHAAIIGSNGSYNGITSNVKGTFVKMSSSTNNADTYLEISFTGGTLEPGAHVQIQGRFAKNDWSNYTQSNDYSFKSASQFVEWDQVTAYLNGVLVWGKESR

SEQ ID NO: 86 - protein, 663 aa, synthetic construct
MNHKVHHHHHHIEGRHMSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKGIEENLYFQSHMELGTLEGEFKLGDIEFIKVNKGGGGSGSQVRLVQSGAEVKKSGESLKISCKGSGYSFTSYWIGWVRQMPGKGLEWMGIIYPGDSDTRYSPSFQGQVTISADKSISTVYLQWSSLKASDTAMYYCARQWSHYTYDYYYGGGGSGGGGSGGGGSGGGGSGGGGSQSVLTQPASVSGSPGQSITISCTGISSDVGGYNSVSWYQQHPGKAPKLMIYDVTNRPSGVSNRFSGSKSGNTASLTISGLQAEDEADYYCSSYTVDVSGNLKVEFYNSNPSDTTNSINPQFKVTNTGSSAIDLSKLTLRYYYTVDGQKDQTFWCDHAAIIGSNGSYNGITSNVKGTFVKMSSSTNNADTYLEISFTGGTLEPGAHVQIQGRFAKNDWSNYTQSNDYSFKSASQFVEWDQVTAYLNGVLVWGKESR

SEQ ID NO: 87 - protein, 671 aa, synthetic construct
MNHKVHHHHHHIEGRHMSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKGIEENLYFQSHMELGTLEGEFDPIVMIDNDKPITGGGGSGSQVQLVESGGVVVQPGGSLRLSCAASGFTFHDHTMHWVRQAPGKGLEWVSLITWNGGTIHYSDSVKGRFTISRDNSKNSLYLQMNSLRTEDTALYYCAKDLGRGGWYLPSDAGGGGSGGGGSGGGGSGGGGSGGGGSQSVLTQPPSASGTPGQRVTISCSGSSANIGSNTVNWYQHLPGTAPKLLIYSNNQRPSGVPDRFSGSKSGTSASLAISGLQSEDEADYYCAAWDDSLKGVVDVSGNLKVEFYNSNPSDTTNSINPQFKVTNTGSSAIDLSKLTLRYYYTVDGQKDQTFWCDHAAIIGSNGSYNGITSNVKGTFVKMSSSTNNADTYLEISFTGGTLEPGAHVQIQGRFAKNDWSNYTQSNDYSFKSASQFVEWDQVTAYLNGVLVWGKESR

SEQ ID NO: 88 - protein, 25 aa, synthetic construct [sequence omitted]

SEQ ID NO: 89 - protein, 8 aa, synthetic construct
GGIFNTYT

SEQ ID NO: 90 - protein, 17 aa, synthetic construct
ISWVRQAPGQGLEWMGR

SEQ ID NO: 91 - protein, 8 aa, synthetic construct
IILMSGMA

SEQ ID NO: 92 - protein, 38 aa, synthetic construct
NYAQKIQGRVTITADKSTSTAYMELTSLRSDDTAVYYC

SEQ ID NO: 93 - protein, 20 aa, synthetic construct
ARGFNGNYYGWGDDDAFDIW

SEQ ID NO: 94 - protein, 25 aa, synthetic construct
QTVLTQPPSVSGAPGQRVTISCTGS

SEQ ID NO: 95 - protein, 9 aa, synthetic construct
NSNIGAGYD

SEQ ID NO: 96 - protein, 17 aa, synthetic construct
VHWYQQLPGTAPKLLIC

SEQ ID NO: 97 - skipped

SEQ ID NO: 98 - protein, 36 aa, synthetic construct
NRPSGVPDRFSGSKSGTSASLAITGLQAEDEADYYC

SEQ ID NO: 99 - protein, 13 aa, synthetic construct
QSYDSSLSGPNWV

SEQ ID NO: 100 - protein, 25 aa, synthetic construct
QVRLVQSGAEVKKSGESLKISCKGS

SEQ ID NO: 101 - protein, 10 aa, synthetic construct
GFSLSTSGVG

SEQ ID NO: 102 - protein, 17 aa, synthetic construct
IGWVRQMPGKGLEWMGI

SEQ ID NO: 103 - protein, 8 aa, synthetic construct
IYPGDSDT

SEQ ID NO: 104 - protein, 38 aa, synthetic construct
RYSPSFQGQVTISADKSISTVYLQWSSLKASDTAMYYC

SEQ ID NO: 105 - protein, 13 aa, synthetic construct
ARQWSHYTYDYYY

SEQ ID NO: 106 - protein, 23 aa, synthetic construct
QSVLTQPASVSGSPGQSITISCT

SEQ ID NO: 107 - protein, 11 aa, synthetic construct
GISSDVGGYNS

SEQ ID NO: 108 - protein, 17 aa, synthetic construct
VSWYQQHPGKAPKLMIY

SEQ ID NO: 109 - skipped

SEQ ID NO: 110 - protein, 36 aa, synthetic construct
NRPSGVSNRFSGSKSGNTASLTISGLQAEDEADYYC

SEQ ID NO: 111 - protein, 4 aa, synthetic construct
SSYT

SEQ ID NO: 112 - protein, 25 aa, synthetic construct
QVQLVESGGVVVQPGGSLRLSCAAS

SEQ ID NO: 113 - protein, 8 aa, synthetic construct
GFTFHDHT

SEQ ID NO: 114 - protein, 17 aa, synthetic construct
MHWVRQAPGKGLEWVSL

SEQ ID NO: 115 - protein, 8 aa, synthetic construct
ITWNGGTI

SEQ ID NO: 116 - protein, 38 aa, synthetic construct
HYSDSVKGRFTISRDNSKNSLYLQMNSLRTEDTALYYC

SEQ ID NO: 117 - protein, 15 aa, synthetic construct
AKDLGRGGWYLPSDA

SEQ ID NO: 118 - protein, 25 aa, synthetic construct
QSVLTQPPSASGTPGQRVTISCSGS

SEQ ID NO: 119 - protein, 8 aa, synthetic construct
SANIGSNT

SEQ ID NO: 120 - protein, 17 aa, synthetic construct
VNWYQHLPGTAPKLLIY

SEQ ID NO: 121 - skipped

SEQ ID NO: 122 - protein, 36 aa, synthetic construct
QRPSGVPDRFSGSKSGTSASLAISGLQSEDEADYYC

SEQ ID NO: 123 - protein, 10 aa, synthetic construct
AAWDDSLKGV

SEQ ID NO: 124 - protein, 51 aa, synthetic construct
TNPGVSAWQVNTAYTAGQLVTYNGKTYKCLQPHTSLAGWEPSNVPALWQLQ

SEQ ID NO: 125 - protein, 44 aa, synthetic construct
YSRCQLQGFNCVVRSYGLPTIPCCRGLTCRSYFPGSTYGRCQRY

SEQ ID NO: 126 - protein, 588 aa, synthetic construct
MNHKVHHHHHHIEGRHMSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKGIEENLYFQSHMELGTLEGEFDPIVMIDNDKPITGGGGSGSQVQLQESGPGLVKPSETLSLTCTVSGYSISSGYYWGWIRQPPGKGLEWIGSIYHSGSTYYNPSLKTRVTISVDTSKNQFSLKLSSVTAADTAVYYCARAVVGIVVVPAAGRRAFDIWGQGTMVTVSSGGGGSGGGGSGGGGSGGGGSGGGGSQSALTQPPSASGSPGQSVTISCTGTSSDVGGYNYVSWYQQHPGKAPKLMIYEVSKRPSGVPDRFSGSKSGNTASLTVSGLQAEDEADYYCSSYAGSNNLVFGGGTKVDTNPGVSAWQVNTAYTAGQLVTYNGKTYKCLQPHTSLAGWEPSNVPALWQLQSR

SEQ ID NO: 127 - protein, 588 aa, synthetic construct; identical to SEQ ID NO: 126

SEQ ID NO: 128 - protein, 581 aa, synthetic construct
MNHKVHHHHHHIEGRHMSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKGIEENLYFQSHMELGTLEGEFDPIVMIDNDKPITGGGGSGSQVQLQESGPGLVKPSETLSLTCTVSGYSISSGYYWGWIRQPPGKGLEWIGSIYHSGSTYYNPSLKTRVTISVDTSKNQFSLKLSSVTAADTAVYYCARAVVGIVVVPAAGRRAFDIWGQGTMVTVSSGGGGSGGGGSGGGGSGGGGSGGGGSQSALTQPPSASGSPGQSVTISCTGTSSDVGGYNYVSWYQQHPGKAPKLMIYEVSKRPSGVPDRFSGSKSGNTASLTVSGLQAEDEADYYCSSYAGSNNLVFGGGTKVDYSRCQLQGFNCVVRSYGLPTIPCCRGLTCRSYFPGSTYGRCQRYSR

SEQ ID NO: 129 - protein, 581 aa, synthetic construct; identical to SEQ ID NO: 128

SEQ ID NO: 130 - protein, 131 aa, synthetic construct
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGL

SEQ ID NO: 131 - protein, 131 aa, synthetic construct
QSTIEEQAKTFLDFFNIQAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGL

SEQ ID NO: 132 - protein, 131 aa, synthetic construct
QSTIEEQAYTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGL

SEQ ID NO: 133 - protein, 131 aa, synthetic construct
QSTIEEQAYTFLDKFNHEAEDLFYQSSLASWNYNTNITEENVQNMNNAGDKWSAFLKEQSTTAQMYPLQEIQNLTVKLQLQALQQNGSSVLSEDKSKRLNTILNTMSTIYSTGKVCNPDNPQECLLLEPGL

SEQ ID NO: 134 - protein, 131 aa, synthetic construct
QSTIEEQAYTFLDKFNHEAEDLFYQSSLASWNYNTNITEENVQNMNNAGDKWSAFLKEQSTLAQMYPLQEIQNLQVKLQLQALQQNGSSVLSEDKSKRLNTILNTMSTIYSTGKVCNPDNPQECLLLEPGL

SEQ ID NO: 135 - protein, 131 aa, synthetic construct
QSTIEEQAYTFLDKFNAEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGL

SEQ ID NO: 136 - protein, 547 aa, synthetic construct
MNHKVHHHHHHIEGRHMSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKGIEENLYFQSHMELGTLEGEFQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLVDVSGNLKVEFYNSNPSDTTNSINPQFKVTNTGSSAIDLSKLTLRYYYTVDGQKDQTFWCDHAAIIGSNGSYNGITSNVKGTFVKMSSSTNNADTYLEISFTGGTLEPGAHVQIQGRFAKNDWSNYTQSNDYSFKSASQFVEWDQVTAYLNGVLVWGKESR

SEQ ID NO: 137 - protein, 547 aa, synthetic construct
MNHKVHHHHHHIEGRHMSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKGIEENLYFQSHMELGTLEGEFQSTIEEQAKTFLDFFNIQAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLVDVSGNLKVEFYNSNPSDTTNSINPQFKVTNTGSSAIDLSKLTLRYYYTVDGQKDQTFWCDHAAIIGSNGSYNGITSNVKGTFVKMSSSTNNADTYLEISFTGGTLEPGAHVQIQGRFAKNDWSNYTQSNDYSFKSASQFVEWDQVTAYLNGVLVWGKESR

SEQ ID NO: 138 - protein, 547 aa, synthetic construct
MNHKVHHHHHHIEGRHMSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKGIEENLYFQSHMELGTLEGEFQSTIEEQAYTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLVDVSGNLKVEFYNSNPSDTTNSINPQFKVTNTGSSAIDLSKLTLRYYYTVDGQKDQTFWCDHAAIIGSNGSYNGITSNVKGTFVKMSSSTNNADTYLEISFTGGTLEPGAHVQIQGRFAKNDWSNYTQSNDYSFKSASQFVEWDQVTAYLNGVLVWGKESR

SEQ ID NO: 139 - protein, 547 aa, synthetic construct
MNHKVHHHHHHIEGRHMSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKGIEENLYFQSHMELGTLEGEFQSTIEEQAYTFLDKFNHEAEDLFYQSSLASWNYNTNITEENVQNMNNAGDKWSAFLKEQSTTAQMYPLQEIQNLTVKLQLQALQQNGSSVLSEDKSKRLNTILNTMSTIYSTGKVCNPDNPQECLLLEPGLVDVSGNLKVEFYNSNPSDTTNSINPQFKVTNTGSSAIDLSKLTLRYYYTVDGQKDQTFWCDHAAIIGSNGSYNGITSNVKGTFVKMSSSTNNADTYLEISFTGGTLEPGAHVQIQGRFAKNDWSNYTQSNDYSFKSASQFVEWDQVTAYLNGVLVWGKESR

SEQ ID NO: 140 - protein, 547 aa, synthetic construct
MNHKVHHHHHHIEGRHMSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKGIEENLYFQSHMELGTLEGEFQSTIEEQAYTFLDKFNHEAEDLFYQSSLASWNYNTNITEENVQNMNNAGDKWSAFLKEQSTLAQMYPLQEIQNLQVKLQLQALQQNGSSVLSEDKSKRLNTILNTMSTIYSTGKVCNPDNPQECLLLEPGLVDVSGNLKVEFYNSNPSDTTNSINPQFKVTNTGSSAIDLSKLTLRYYYTVDGQKDQTFWCDHAAIIGSNGSYNGITSNVKGTFVKMSSSTNNADTYLEISFTGGTLEPGAHVQIQGRFAKNDWSNYTQSNDYSFKSASQFVEWDQVTAYLNGVLVWGKESR

SEQ ID NO: 141 - protein, 547 aa, synthetic construct
MNHKVHHHHHHIEGRHMSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKGIEENLYFQSHMELGTLEGEFQSTIEEQAYTFLDKFNAEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLVDVSGNLKVEFYNSNPSDTTNSINPQFKVTNTGSSAIDLSKLTLRYYYTVDGQKDQTFWCDHAAIIGSNGSYNGITSNVKGTFVKMSSSTNNADTYLEISFTGGTLEPGAHVQIQGRFAKNDWSNYTQSNDYSFKSASQFVEWDQVTAYLNGVLVWGKESR

(19) United States
(12) Patent Application Publication
(10) Pub. No.: US 2024/0376182 A1
(43) Pub. Date: Nov. 14, 2024

(54) ANTI-VIRUS MOIETY IMMOBILISED IN A MATRIX

(71) Applicant: SAMATVA RESEARCH CORPORATION, Thunder Bay (CA)

(72) Inventors: Sanjoy Kumar Gupta, Thunder Bay (CA); Neelam Khaper, Thunder Bay (CA)

(73) Assignee: SAMATVA RESEARCH CORPORATION, Thunder Bay (CA)

(21) Appl. No.: 18/690,098

(22) PCT Filed: Sep. 9, 2022
(86) PCT No.: PCT/GB2022/052291

§ 371 Date: Mar. 7, 2024

(30) Foreign Application Priority Data
Sep. 10, 2021 (EP) 21195979.6

(51) Int. Cl. C07K 16/10; A01N 63/50; A01P 1/00; A61K 8/64; A61L 2/00; A61L 2/18; A61L 2/23; A61L 9/00; A61L 101/32; A61Q 17/00; A62B 23/02

(52) U.S. Cl. CPC C07K 16/1003; A01N 63/50; A01P 1/00; A61K 8/64; A61L 2/0088; A61L 2/18; A61L 2/23; A61L 9/00; A61Q 17/005; A61L 2101/32; A61L 2209/14; A62B 23/025; C07K 2317/622

(57) ABSTRACT

The invention relates to hygiene products for filtering or removal of virus particles and/or fragments thereof using an anti-virus moiety immobilised in a matrix.

### FIELD OF THE INVENTION

The invention relates to products for the removal of virus particles and/or fragments thereof, and methods and uses thereof.

### BACKGROUND TO THE INVENTION

To survive within a population, viruses must spread from an infected host to a susceptible host in a process known as viral transmission. Although they cannot replicate independently outside a host, virus particles can remain infectious outside the body and persist in the air and on surfaces for up to several hours. Transmission can be facilitated by, for example, respiratory droplets, airborne particles, and contaminated surfaces known as fomites.

Respiratory droplets are expelled when an infected person coughs or sneezes, for example. Respiratory droplets are approximately 5-10 μm in diameter and transmission can occur when a person is in close contact with an infected host.

Small droplets of less than 5 μm in diameter (often known as droplet nuclei or aerosols) are responsible for airborne transmission. These airborne particles remain suspended in air for significant periods of time and can readily be inhaled.

Additionally, respiratory droplets and airborne particles expelled by infected individuals can contaminate surfaces and objects, creating fomites. Viral transmission may therefore occur indirectly through touching such fomites, followed by touching the mouth, nose or eyes.

Strategies to reduce the transmission of viruses include the use of filters, masks and surface cleaners. HEPA filters and N95 masks are designed to provide protection against both respiratory droplets and airborne particles. They block approximately 99.9% of all particles that are at least 0.3 μm in diameter, and they are thought to be capable of blocking approximately 99.8% of particles that are at least 0.1 μm in diameter. N95 masks can also reduce the inhalation of airborne virus particles and droplets into the user's airway. Additionally, surface cleaners and hand sanitisers are effective at dissolving the viral lipid coat, thus preventing transmission by eliminating viruses from fomites.

The importance of virus transmission preventative strategies is highlighted in the coronavirus disease 2019 (COVID-19) pandemic, which is a severe acute respiratory syndrome caused by SARS-COV-2 and has posed unprecedented challenges to public health on a global scale. With the emergence of variant SARS-COV-2 strains which can spread even more widely and quickly than the original SARS-COV-2 strain, there is an urgent need to identify effective preventative measures to reduce virus transmission.

As demonstrated during the COVID-19 pandemic, filters, masks, surface cleaners and hand sanitisers each have limitations. For example, HEPA filters and N95 masks are expensive and need to be replaced frequently. In particular, N95 masks and surgical masks are also designed for single-use and are environmentally unfriendly. N95 masks containing exhalation valves allow exhaled air to be propelled outwards, meaning that particles in exhaled air travel further than if a regular (paper or cotton) mask were used. Additionally, mechanical action caused by speaking, shouting, sneezing or coughing can cause significant emission of virus particles from the fibres of regular masks. Regular masks and N95 masks are also often worn incorrectly, and can give users a false sense of security. Furthermore, the waterproof nature of the N95 and surgical masks leads to perspiration build up, reducing wearer comfort. Surface cleaners and alcohol-based hand sanitisers contain chemicals that are toxic when misused, with the latter leading to potential long-term health effects, such as increased chance of antimicrobial resistance.

It is therefore an object of the invention to identify further and improved methods and products for removing or filtering virus particles and/or fragments thereof, e.g. from the air or from fomites.

It is also an object of the invention to identify further and improved methods and products for preventing the transmission of viruses, such as SARS-COV-2.

### SUMMARY OF THE INVENTION

Anti-virus moieties immobilised in hygiene products can be used to specifically “trap” target virus particles and/or fragments thereof. Hence, such immobilised anti-virus moieties are particularly useful for preventing virus transmission, e.g. by filtering or removing virus particles and/or fragments thereof from, e.g. the environment or a surface.

The hygiene products and components thereof can be easily prepared in an economic fashion, are non-toxic and reusable.

As shown in the Examples, anti-virus moieties, when immobilised in a matrix, are effective in binding to virus pseudoparticles with high affinity and for an extended period of time. In particular, an anti-virus moiety was prepared in the form of a single-chain variable fragment (scFv) derived from human monoclonal antibody (mAb) which recognises the receptor binding domain (RBD) of the spike protein in SARS-COV-2. The anti-virus moiety was coupled to a cellulose-binding domain, a chitin-binding domain, or a polyester affinity tag. In some examples, the anti-virus moiety was prepared in the form of a fragment derived from a receptor or mutant thereof which recognises the receptor binding domain (RBD) of the spike protein in SARS-COV-2 (decoy receptor). The anti-virus moiety was coupled to a cellulose-binding domain. The fusion proteins used in the Examples are referred to herein as Karunyam proteins.

Each of the Karunyam proteins used in the Examples was found to specifically bind to the RBD of the spike protein. The binding affinity between each of the Karunyam proteins and the RBD was found to be similar to the binding affinity between the ACE2 receptor and the RBD, and the binding affinity between the monoclonal antibodies from which the scFvs were derived and the RBD.

Furthermore, when the Karunyam proteins were immobilised on cellulose beads or polyester fibres, they were effective in binding to virus pseudoparticles (RBD-spike protein conjugated to FITC nanoparticles) quickly and the binding remained intact even after one week. The Karunyam proteins immobilised on cellulose beads or polyester fibres were also capable of retaining the virus pseudoparticles after centrifugation at high speed.

Furthermore, virus pseudoparticles trapped on Karunyam proteins immobilised on polyester fibres were substantially removed after incubation with water or detergent solutions at 100° C. Additionally, polyester fibres can be reused to trap new virus pseudoparticles after said removal with water or detergent solutions at 100° C.

These findings therefore confirm the effectiveness of anti-virus moieties, when immobilised on a matrix, in binding to virus particles, and hence their usefulness as hygiene products for preventing virus transmission, e.g. by filtering and removing viruses from the environment or a surface.

Accordingly, the invention provides an anti-virus moiety immobilised in a matrix, wherein the matrix is suitable for use as a hygiene product.

The invention also provides a fusion protein comprising: (i) an anti-virus moiety, such as an anti-virus moiety described herein, and (ii) a matrix-binding domain; optionally wherein the fusion protein further comprises a linker between the anti-virus moiety and the matrix-binding domain.

The invention also provides a matrix comprising the anti-virus moiety or the fusion protein of the invention immobilised therein, optionally wherein the matrix is cellulose, starch, collagen, insect shells, mucous membranes, or an inorganic matrix.

The invention also provides a hygiene product comprising the anti-virus moiety, the fusion protein or the matrix of the invention.

The invention also provides a method of filtering or removing virus particles and/or fragments thereof from the environment or a surface, comprising applying the immobilised anti-virus moiety, the fusion protein, the matrix, or the hygiene product of the invention to filter or remove virus particles and/or fragments thereof from the environment or a surface.

The invention also provides the use of the immobilised anti-virus moiety, the fusion protein, the matrix, or the hygiene product of the invention to filter or remove virus particles and/or fragments thereof from the environment or a surface.

The invention also provides a method of preventing virus transmission comprising filtering or removing virus particles and/or fragments thereof from the environment or a surface according to the method or use of the invention.

### BRIEF DESCRIPTION OF THE FIGURES

FIG. 1 shows a 4-12% SDS-PAGE of proteins expressed from bacteria containing a vector encoding Karunyam 9. Lane 1 shows molecular weight markers. Lane 2 is whole cell lysate prior to induction of vector expression. Lanes 3-6 are whole cell lysates of four preparations following induction of vector expression. Lanes 7-10 are four separate preparations of Karunyam 9 following purification. Arrows indicate the Karunyam 9 protein.

FIG. 2 shows that anti-virus Karunyam proteins can bind to the RBD-Spike protein with high specificity and high affinity. A is a Far-Western blot of: (1) serial dilutions of commercial RBD-Spike binding mouse monoclonal antibodies; and (2)-(4) Karunyam proteins probed with RBD-Spike protein conjugated to HRP. B is a graph depicting the results of an ELISA with immobilised Karunyam 9 probed with RBD-Spike protein conjugated to HRP.

FIG. 3 shows that Karunyam 9 immobilised on cellulose beads bind to RBD-Spike protein conjugated to FITC nanoparticles (virus pseudoparticles) for extended periods of time. A and B shows the cellulose beads under light and fluorescence microscopy respectively, after 5 minutes. C and D shows the cellulose beads under light and fluorescence microscopy respectively, after one week in solution.

FIG. 4 shows that Karunyam proteins immobilised on cellulose beads retain virus pseudoparticles with high affinity. The amount of virus pseudoparticles bound to each immobilised Karunyam protein was estimated by subtracting the unbound fraction from the total virus pseudoparticle as estimated by immunochromatography strip analysis. The unbound fraction was estimated using a competitive ELISA.

FIG. 5 shows that Karunyam 13 immobilised on cellulose beads binds to the Delta RBD-Spike protein conjugated to FITC nanoparticles (virus pseudoparticles) for extended periods of time. A and B shows the cellulose beads under fluorescence microscopy after 5 minutes and after one week respectively, in solution at room temperature.

FIG. 6 shows that Karunyam 13 immobilised on cellulose beads binds to the Delta-plus RBD-Spike protein conjugated to FITC nanoparticles (virus pseudoparticles) for extended periods of time. A and B shows the cellulose beads under fluorescence microscopy after 5 minutes after 5 minutes and after one week respectively, in solution at room temperature.

FIG. 7 shows that Karunyam 23 immobilised on polyester fibre binds to the RBD-Spike protein conjugated to FITC nanoparticles (virus pseudoparticles). A and B shows the polyester fibre under light and fluorescence microscopy respectively, after 5 minutes. C and D shows the polyester fibre under light and fluorescence microscopy respectively, after one week in solution.

FIG. 8 shows that FITC nanoparticles (virus pseudoparticles) bound to Karunyam 23 immobilised on polyester fibre can be partially removed by incubating the fibre at 37° C. with different detergent solutions. A shows the polyester fibre under fluorescence microscopy before incubation with detergent solution. B-E show the polyester fibre under fluorescence microscopy after one hour at 37° C. in 2% Sodium lauroyl sarcosinate, 2% SDS, 2% Triton X-100, 2% Tween-20, respectively.

FIG. 9 shows that FITC nanoparticles (virus pseudoparticles) bound to Karunyam 23 immobilised on polyester fibre can be substantially removed by incubating the fibre at 100° C. with different detergent solutions. A shows the polyester fibre under fluorescence microscopy before incubation with detergent solution. B-E show the polyester fibre under fluorescence microscopy after one hour at 100° C. in 2% Sodium lauroyl sarcosinate, 2% Triton X-100, 2% Tween-20, distilled water and 10% HOAc, 50% MeOH, respectively.

FIG. 10 shows that polyester fibre that has been removed of substantially all FITC nanoparticles (virus pseudoparticles) after incubating at 100° C. with different detergent solutions can recover virus pseudoparticles after re-incubating with Karunyam 23 and Texas red nanoparticles (virus pseudoparticles).

### DETAILED DESCRIPTION OF THE INVENTION

### Anti-Virus Moiety

An anti-virus moiety useful with the invention is capable of specifically binding to a target virus particle and/or a fragment thereof. Typically, the anti-virus moiety specifically binds to the target virus particle and/or a fragment thereof with high affinity.

By referring to “specifically binding”, it is meant that the anti-virus moiety recognises and binds the target virus and/or a fragment thereof, but does not substantially recognise and bind other molecules, e.g. in a sample such as a biological sample or an environment sample.

The anti-virus moiety may be a protein or a mixture of proteins, but may also be an aptamer, affimer, molecularly imprinted polymer (MIP), or a nucleic acid.

The anti-virus moiety is typically an antibody. The term “antibody” referred to herein may comprise a complete antibody having full length heavy and light chains, or an antigen-binding fragment thereof. For example, the anti-virus moiety may be a full-length antibody.

The anti-virus moiety useful with the invention may be an antigen-binding fragment. An antigen-binding fragment of the invention binds to the same epitope of the parent antibody, i.e. the antibody from which the antigen-binding fragment is derived. An antigen-binding fragment of the invention typically retains the parts of the parent antibody that interact with the epitope. The antigen-binding fragment typically comprises the complementarity-determining regions (CDRs) that interact with the antigen, such as one, two, three, four, five or six CDRs. In some embodiments, the antigen-binding fragment further comprises the structural scaffold surrounding the CDRs of the parent antibody, such as the framework regions (e.g. FR1, 2, or 3) and/or the variable region domains of the heavy and/or light chains. Typically, the antigen-binding fragment retains the same or similar binding affinity to the antigen as the parent antibody.

For example, an antigen-binding fragment may be a Fab, modified Fab, Fab′, modified Fab′, F(ab′)2, Fv, single domain antibody (e.g. VH or VL or VHH), single chain variable fragment (scFv), bi, tri or tetra-valent antibody, Bis-scFv, diabody, triabody, tetrabody or epitope-binding fragments of any of the above (see for example References 1 and 2). The methods for creating and manufacturing antibody fragments are well known in the art (see for example Reference 3).

The inventors found that scFvs are particularly useful with the invention, as shown in the Examples. Hence, the anti-virus moiety useful with the invention may be a scFv.

The anti-virus moiety useful with the invention may be a decoy receptor, such as a decoy receptor comprising a binding domain for SARS-COV-2, e.g. a ACE2 receptor fragment. An ACE2 receptor comprising SEQ ID NO: 130 may be useful with the invention. Modified versions of the ACE2 receptor, e.g. having substitutions at position 27 (e.g. T27Y), 31 (e.g. K31F), 34 (e.g. H34I or H34A), 35 (e.g. E35Q), 79 (e.g. L79T) and/or 92 (e.g. T92Q) of SEQ ID NO: 130, are particularly useful with the invention. These mutants have increased binding affinity to SARS-COV-2 RBD-spike protein (e.g. see Reference 4). Examples of useful modified ACE2 receptors are provided in SEQ ID NOs: 131 to 135.

An anti-virus moiety useful with the invention (e.g. a decoy receptor) may comprise an ACE2 receptor fragment as set out in SEQ ID NO: 130, 131, 132, 133, 134 or 135. The anti-virus moiety (e.g. a decoy receptor) may comprise or consist of an amino acid sequence having ≥70% (i.e. 70% or more), ≥80%, ≥90%, ≥95%, ≥96%, ≥97%, ≥98%, ≥99% or 100% sequence identity with SEQ ID NO: 130, 131, 132, 133, 134 or 135, provided that it is capable of specifically binding to the target virus particle and/or fragment thereof. The anti-virus moiety may bind to any epitope in a target virus particle. For example, it may bind to a neutralising epitope of the target virus particle, e.g. it may neutralise the activity of the virus. Alternatively, the anti-virus moiety may bind to a non-neutralising epitope.

The target virus particle may be coronavirus, Ebola virus, respiratory syncytial virus (RSV), influenza virus, adenovirus, human rhinovirus, human metapneumovirus, parainfluenza virus, hantavirus, Bocavirus, or Marburg virus. For example, the target virus particle may be coronavirus, e.g. SARS-COV-2, SARS-COV-1 or Middle Eastern Respiratory Syndrome (MERS).

The target virus particle may be SARS-COV-2. SARS-COV-2 is a beta coronavirus, which is related to SARS-COV and MERS coronaviruses. Embedded in the membrane envelope of both SARS-COV-2 and SARS-COV-1 viruses are multiple copies of a glycoprotein known as the spike protein, which recognises the angiotensin converting enzyme 2 (ACE2) receptor on host cells to facilitate cell entry. The specific region of the spike protein that interacts with the ACE2 receptor, and therefore the region responsible for attaching the virus to host cells, is known as the receptor binding domain (RBD).

The target virus particle may be any SARS-COV-2 strain, such as a member of lineage Alpha (B.1.1.7 and Q lineages), Beta (B.1.351 and descendant lineages), Gamma (P.1 and descendent lineages), Delta (B.1.617.2 and AY lineages), Epsilon (B.1.427 and B.1.1429), Eta (B.1.525), Iota (B.1.526), Kappa (B.1.617.1), 1.617.3, Mu (B.1.621 and B.1.621.1), Zeta (P.2), or Omicron.

The epitope in the target virus particle may be in the spike protein of a coronavirus, e.g. SARS-COV-2.

The epitope may be in the the S1 subunit of the spike protein, such as the receptor binding domain (RBD) or N-terminal domain (NTD). For example, the epitope may be in the RBD of the spike protein.

The anti-virus moiety may be a scFv that binds to the RBD in the spike protein of SAR-COV-2.

Known antibodies in the art that specifically bind to coronavirus, e.g. SARS-COV-2, are useful with the invention. For example, antibodies P2B-2F6, P2C-1F11, P2C-1A3, P2C-1D5, P2A-1A10, P2A-1B3, P2C-1C8, P2A-1A8, P2C-1E1, P2A-1A9, P2C-1C10, P2B-2G4, P2B-2G11, P1A-1C10, PIA-1C7, PIA-1D1, PIA-1B2, PIA-1C1, m396, 80R, CR3022, SARS 2-38, CT-P59, LY-CoV1404, S2X259, S2H97, and S2K146 as disclosed in References 5, 6, 7, 8, 9, 15, 16 and 17 are useful with the invention. For example, the anti-virus moiety useful with the invention may be any of these antibodies, a fragment thereof, and/or a molecule derived therefrom.

For example, the anti-virus moiety may be derived from P2B-2F6, P2C-1F11, P2C-1A3, SARS 2-38, CT-P59, LY-CoV1404, S2X259, S2H97 or S2K146 such as the scFvs in the Examples.

Hence, an anti-virus moiety useful with the invention (e.g. an antibody) may comprise the complementary determining regions: CDRH1, CDRH2, CDRH3, CDRL1, CDRL2 and CDRL3 as set out in SEQ ID NOs: 11, 13, 15, 17, 19 and 21 respectively. The anti-virus moiety may additionally comprise the framework regions: FRH1, FRH2, FRH3, FRL1, FRL2 and FRL3 as set out in SEQ ID NOs: 10, 12, 14, 16, 18 and 20, respectively. The anti-virus moiety (e.g. an antibody) may comprise or consist of an amino acid sequence having ≥70% (i.e. 70% or more), ≥80%, ≥90%, ≥95%, ≥96%, ≥97%, ≥98%, ≥99% or 100% sequence identity with SEQ ID NO: 1, provided that it is capable of specifically binding to the target virus particle and/or fragment thereof. The anti-virus moiety may be a scFv consisting of SEQ ID NO: 1, i.e. as referred to herein as scFv 9.

An anti-virus moiety useful with the invention (e.g. an antibody) may comprise CDRH1, CDRH2, CDRH3, CDRL1, CDRL2 and CDRL3 as set out in SEQ ID NOs: 23, 25, 27, 29, 31 and 33 respectively. The anti-virus moiety may additionally comprise a FRH1, FRH2, FRH3, FRL1, FRL2 and FRL3 as set out in SEQ ID NOs: 22, 24, 26, 28, 30 and 32, respectively. The anti-virus moiety (e.g. an antibody) may comprise or consist of an amino acid sequence having ≥70% (i.e. 70% or more), ≥80%, ≥90%, ≥95%, ≥96%, ≥97%, ≥98%, ≥99% or 100% sequence identity with SEQ ID NO: 2, provided that it is capable of specifically binding to the target virus particle and/or fragment thereof. The anti-virus moiety may be a scFv consisting of SEQ ID NO: 2, i.e. as referred to herein as scFv 10.

An anti-virus moiety useful with the invention (e.g. an antibody) may comprise CDRH1, CDRH2, CDRH3, CDRL1, CDRL2 and CDRL3 as set out in SEQ ID NOs: 35, 37, 39, 41, 43 and 45 respectively. The anti-virus moiety may additionally comprise a FRH1, FRH2, FRH3, FRL1, FRL2 and FRL3 as set out in SEQ ID NOs: 34, 36, 38, 40, 42 and 44, respectively. The anti-virus moiety may comprise or consist of an amino acid sequence having ≥70% (i.e. 70% or more), ≥80%, ≥90%, ≥95%, ≥96%, ≥97%, ≥98%, ≥99% or 100% sequence identity with SEQ ID NO: 3, provided that it is capable of specifically binding to the target virus particle and/or fragment thereof. The anti-virus moiety may be a scFv consisting of SEQ ID NO: 3, i.e. as referred to herein as scFv 11.

An anti-virus moiety useful with the invention (e.g. an antibody) may comprise CDRH1, CDRH2, CDRL1 and CDRL2 as set out in SEQ ID NOs: 53, 55, 58 and 60, respectively. The anti-virus moiety may additionally comprise a FRH1, FRH2, FRH3, FRL1, FRL2 and FRL3 as set out in SEQ ID NOs: 52, 54, 56, 57, 59 and 61, respectively. The anti-virus moiety may comprise or consist of an amino acid sequence having ≥70% (i.e. 70% or more), ≥80%, ≥90%, ≥95%, ≥96%, ≥97%, ≥98%, ≥99% or 100% sequence identity with SEQ ID NO: 46, provided that it is capable of specifically binding to the target virus particle and/or fragment thereof. The anti-virus moiety may be a scFv consisting of SEQ ID NO: 46, i.e. as referred to herein as scFv 13.

An anti-virus moiety useful with the invention (e.g. an antibody) may comprise CDRH1, CDRH2, CDRL1 and CDRL2 as set out in SEQ ID NOs: 63, 65, 68 and 70, respectively. The anti-virus moiety may additionally comprise a FRH1, FRH2, FRH3, FRL1, FRL2 and FRL3 as set out in SEQ ID NOs: 62, 64, 66, 67, 69 and 71, respectively. The anti-virus moiety may comprise or consist of an amino acid sequence having ≥70% (i.e. 70% or more), ≥80%, ≥90%, ≥95%, ≥96%, ≥97%, ≥98%, ≥99% or 100% sequence identity with SEQ ID NO: 47, provided that it is capable of specifically binding to the target virus particle and/or fragment thereof. The anti-virus moiety may be a scFv consisting of SEQ ID NO: 47, i.e. as referred to herein as scFv 14.

An anti-virus moiety useful with the invention (e.g. an antibody) may comprise CDRH1, CDRH2, CDRL1 and CDRL2 as set out in SEQ ID NOs: 73, 75, 78 and 80, respectively. The anti-virus moiety may additionally comprise a FRH1, FRH2, FRH3, FRL1, FRL2 and FRL3 as set out in SEQ ID NOs: 72, 74, 76, 77, 79 and 81, respectively. The anti-virus moiety may comprise or consist of an amino acid sequence having ≥70% (i.e. 70% or more), ≥80%, ≥90%, ≥95%, ≥96%, ≥97%, ≥98%, ≥99% or 100% sequence identity with SEQ ID NO: 48, provided that it is capable of specifically binding to the target virus particle and/or fragment thereof. The anti-virus moiety may be a scFv consisting of SEQ ID NO: 48, i.e. as referred to herein as scFv 15.

An anti-virus moiety useful with the invention (e.g. an antibody) may comprise CDRH1, CDRH2, CDRH3, CDRL1, CDRL2 and CDRL3 as set out in SEQ ID NOs: 89, 91, 93, 95, 97 and 99 respectively. The anti-virus moiety may additionally comprise a FRH1, FRH2, FRH3, FRL1, FRL2 and FRL3 as set out in SEQ ID NOs: 88, 90, 92, 94, 96 and 98, respectively. The anti-virus moiety may comprise or consist of an amino acid sequence having ≥70% (i.e. 70% or more), ≥80%, ≥90%, ≥95%, ≥96%, ≥97%, ≥98%, ≥99% or 100% sequence identity with SEQ ID NO: 82, provided that it is capable of specifically binding to the target virus particle and/or fragment thereof. The anti-virus moiety may be a scFv consisting of SEQ ID NO: 82, i.e. as referred to herein as scFv 18.

An anti-virus moiety useful with the invention (e.g. an antibody) may comprise CDRH1, CDRH2, CDRH3, CDRL1, CDRL2 and CDRL3 as set out in SEQ ID NOs: 101, 103, 105, 107, 109 and 111 respectively. The anti-virus moiety may additionally comprise a FRH1, FRH2, FRH3, FRL1, FRL2 and FRL3 as set out in SEQ ID NOs: 100, 102, 104, 106, 108 and 110, respectively. The anti-virus moiety may comprise or consist of an amino acid sequence having ≥70% (i.e. 70% or more), ≥80%, ≥90%, ≥95%, ≥96%, ≥97%, ≥98%, ≥99% or 100% sequence identity with SEQ ID NO: 83, provided that it is capable of specifically binding to the target virus particle and/or fragment thereof. The anti-virus moiety may be a scFv consisting of SEQ ID NO: 83, i.e. as referred to herein as scFv 19.

An anti-virus moiety useful with the invention (e.g. an antibody) may comprise CDRH1, CDRH2, CDRH3, CDRL1, CDRL2 and CDRL3 as set out in SEQ ID NOs: 113, 115, 117, 119, 121 and 123 respectively. The anti-virus moiety may additionally comprise a FRH1, FRH2, FRH3, FRL1, FRL2 and FRL3 as set out in SEQ ID NOs: 112, 114, 116, 118, 120 and 122, respectively. The anti-virus moiety may comprise or consist of an amino acid sequence having ≥70% (i.e. 70% or more), ≥80%, ≥90%, ≥95%, ≥96%, ≥97%, ≥98%, ≥99% or 100% sequence identity with SEQ ID NO: 84, provided that it is capable of specifically binding to the target virus particle and/or fragment thereof. The anti-virus moiety may be a scFv consisting of SEQ ID NO: 84, i.e. as referred to herein as scFv 20. Typically, the anti-virus moiety binds to the epitope, e.g. in the RBD of SARS-COV-2, with high affinity, e.g. with a K_{D} value of ≤100 nM, ≤50 nM, ≤10 nM, ≤5 nM, ≤1 nM, ≤0.5 nM, or ≤0.1 nM. For example, the K_{D} value may be between 1 to 10 nM, e.g. between 4 nM to 8 nM. For example, the K_{D} values of some of the anti-virus moieties of the invention are provided in Tables 3 and 4. Binding affinity (K_{D}) can be analysed by any suitable means known in the art, for example, by ELISA or Surface Plasmon Resonance, i.e., detection of real-time biospecific interactions by detection of changes in protein concentration in a biosensor matrix using, for example, the BIACORE system (Pharmacia Biosensor AB, Uppsala, Sweden).

The anti-virus moiety is typically prepared in an expression system. Expression systems useful for protein production are known in the art, e.g. mammalian (e.g. CHO cells, 293 cells), insect, yeast, bacterial (e.g. E. coli), algal, and cell-free protein expression systems, and any of these expression systems can be used to express an anti-virus moiety useful with the invention.

The anti-virus moiety is typically soluble in the expression system, e.g. E. coli. For example, the anti-virus moiety may be naturally soluble in the physiological condition of the expression system, e.g. in E. coli cells. The anti-virus moiety may be fused to a solubility tag to increase its solubility in the physiological condition of the expression system. Useful solubility tags are known in the art, e.g. Glutathione-S-transferase (GST), Maltose Binding Protein (MBP) and Thioredoxin (TRx), and any of these solubility tags can be used to express an anti-virus moiety useful with the invention. The solubility tag may be fused to the N- or C-terminal of anti-virus moiety, for example.

For example, the anti-virus moiety may be fused to GST comprising or consisting of an amino acid sequence having ≥70% (i.e. 70% or more), ≥80%, ≥90%, ≥95%, ≥96%, ≥97%, ≥98%, ≥99% or 100% sequence identity with SEQ ID NO: 5.

It is within the teaching herein that the anti-virus moiety may be fused to an affinity tag for protein purification e.g. HA-tag which correspond to amino acids 98 to 106 of human influenza hemagglutinin, polyhistidine (His) (SEQ ID NO: 4), c-myc and FLAG. The affinity tag may be fused to the N- or C-terminal of anti-virus moiety, for example.

Hence, the invention also provides a fusion protein comprising: (i) an anti-virus moiety described herein and (ii) a solubility tag and/or an affinity tag. The solubility tag may be GST comprising or consisting of an amino acid sequence having ≥70% (i.e. 70% or more), ≥80%, ≥90%, ≥95%, ≥96%, ≥97%, ≥98%, ≥99% or 100% sequence identity with SEQ ID NO: 5. The affinity tag may be polyhistidine comprising or consisting of SEQ ID NO: 4. The solubility tag and/or the affinity tag may be fused to the N- or C-terminal of anti-virus moiety. For example, the fusion protein may comprise the solubility tag and the affinity tag fused to the N-terminal of anti-virus moiety.

The anti-virus moiety is typically functional in the expression system, e.g. E. coli. For example, the anti-virus moiety is capable of specifically binding to its target, e.g. with high affinity as described herein. The anti-virus moiety expressed from the expression system may be modified and/or manipulated (e.g. subjected to a denaturation and renaturation protocol) to obtain a functional anti-virus moiety.

Hence, the invention also provides a method of preparing an anti-virus moiety, comprising fusing the anti-virus moiety with a solubility tag and optionally subjecting the anti-virus moiety to a denaturation and renaturation protocol.

Methods of testing the functional properties of an anti-virus moiety include determining its binding affinity to the target virus particle. Techniques for determining binding affinity is known in the art, e.g. by ELISA or Surface Plasmon Resonance, i.e., detection of real-time biospecific interactions by detection of changes in protein concentration in a biosensor matrix using, for example, the BIACORE system (Pharmacia Biosensor AB, Uppsala, Sweden).

### The Matrix and the Matrix-Binding Domain

A matrix of the invention is capable of retaining target virus particles and/or fragments thereof via an anti-virus moiety as described herein. The anti-virus moiety useful with the invention is immobilised in a matrix.

The matrix may comprise one or more anti-virus moieties having different specificities. For example, multiple anti-virus moieties having different specificities may be immobilised in the matrix. For example, multiple anti-virus moieties, each specific to a different epitope on the same target virus particle, may be immobilised in the matrix. As a further example, multiple anti-virus moieties, each specific to a different target virus particle may be immobilised in the matrix. Hence, the matrix may be capable of retaining multiple target virus particles.

The matrix may be solid or liquid in nature.

The matrix may be cellulose, starch, collagen, insect shells, mucous membrane, or inorganic matrix.

The cellulose matrix may be a cellulose bead, a paper product, cotton fabric or a cellulose solution.

The anti-virus moiety may be immobilised in a matrix in an irreversible manner. Alternatively, the anti-virus moiety may be immobilised in a matrix in a reversible manner.

The anti-virus moiety may be immobilised by methods well-known in the art, e.g. by cross-linking, covalent binding or physically adsorbing the anti-virus moiety to the matrix.

The anti-virus moiety may be immobilised in the matrix via a matrix-binding domain. For example, the anti-virus moiety may be coupled, e.g. chemically coupled or fused, to a matrix-binding domain without altering the binding capacity of the anti-virus moiety. Hence, the invention provides an anti-virus moiety described herein in the form of a fusion protein comprising an anti-virus moiety and a matrix-binding domain, as explained further below.

The matrix-binding domain is typically a peptide or a mixture of peptides that is capable of specifically binding to a particular matrix.

The matrix-binding domain may bind the matrix with high affinity, e.g. with a K_{D} value of less than 1 μM, e.g. between 10⁻⁶ M to 10⁻¹⁶ M, such as between 10⁻⁶ M to 10⁻¹⁵ M. For example, the K_{D} value is between 0.1 μM to 1 μM.

The anti-virus moiety may be present in a x:y ratio to the matrix-binding domain, where x is between 1 and 10, and y is 1. For example, in the embodiment where the matrix-binding domain is a cellulose-binding domain, the anti-virus moiety may be present in a 1:1 (equimolar) ratio to the cellulose-binding domain. In other embodiments, the ratio may be increased by the specific design to a higher ratio such as 5:1 or 10:1, through multivalent linkage. For example, the use of spider silk with a multivalent linker, such as SpyTag/Spycatcher, would generate a multivalent ratio between the anti-virus moiety and the matrix-binding domain. A higher ratio between an anti-virus moiety and a matrix-binding domain is advantageous because more virus particles and/or fragments thereof may be trapped with a smaller amount of the matrix-binding domain.

The invention also provides a method of immobilising an anti-virus moiety described herein in a matrix. Conditions suitable for the immobilisation are well known in the art and the skilled person would be able to determine such conditions based on teachings in the art.

The matrix-binding domain may be a cellulose-binding domain, a collagen-binding domain, a chitin-binding domain, a mucin-binding domain, or a polymer-binding peptide. Examples of matrix-binding domains and the corresponding matrices are provided in Table 1.

TABLE 1
Examples of matrix-binding domains
and the corresponding matrices.

| Matrix-binding | Examples of matrix- | |
|---|---|---|
| domain | binding domains | Matrix |
| Cellulose-binding | Carbohydrate-binding | Cotton, paper, |
| domain | module (CBM) | cellulose, starch |
| Collagen-binding | | Collagen |
| domain | | |
| Chitin-binding | Chitin-binding | Insect shells, |
| domain | domain | e.g. chitosan |
| Mucin-binding | Galectin 3 carbohydrate | Mucous membrane |
| domain | recognition domain | |
| Polymer binding | Polystyrene (PS) | Polystyrene (PS) |
| peptide | binding peptide | |
| | Polypropylene (PP) | Polypropylene (PP) |
| | binding peptide | (e.g. HEPA filter) |
| | Polyetherimide (PEI) | Polyetherimide (PEI) |
| | binding peptide | |
| | Hyaluronic acid (HA) | Hyaluronic acid (HA) |
| | binding peptide | |
| | Hydroxymethacrylate | Hydroxymethacrylate |
| | (HEMA) binding peptide | (HEMA) |
| | Tachystatin | Polyester (e.g. |
| | affinity tag | polyester fabric sheet) |

As shown in the Examples, the carbohydrate-binding module CBM3 is particularly useful with the invention. Hence, an anti-virus moiety useful with the invention may be coupled (e.g. fused) to CBM3, e.g. comprising SEQ ID NO: 6.

Chitin-binding domain, e.g. of the enzyme chitinase A1 (Bacillus circulans), is particularly useful with the invention, as shown in the Examples. The chitin-binding domain may be added as a tag either at the C- or N-terminus of an anti-virus moiety. The fusion protein may be purified by affinity chromatography with chitin resin, e.g. as described in Reference 7. For example, an anti-virus moiety useful with the invention may be coupled (e.g. fused) to the chitin-binding domain, e.g. comprising SEQ ID NO: 124.

Tachystatin affinity tag, e,g, Tachystatin A2, is particularly useful with the invention, as shown in the Examples. Further examples of tachystatin affinity tag are provided in Reference 8. For example, an anti-virus moiety useful with the invention may be coupled (e.g. fused) to TA2, e.g. comprising SEQ ID NO: 125.

The polymer-binding peptides are useful with the invention as they allow attachment of an anti-virus moiety in a non-covalent fashion to various surfaces such as polystyrene (PS), polypropylene (PP) (e.g. HEPA filter), polyetherimide (PEI), hyaluronic acid (HA) or hydroxymethacrylate (HEMA).

The presence of a matrix-binding domain in the anti-virus moiety according to the invention is particularly advantageous because the matrix binding domain binds with high affinity to the matrix and therefore allows strong attachment in a simple way. Hence, there is no requirement to further modify the matrix and/or the anti-virus moiety for matrix immobilisation (e.g. by chemical treatment), which would have been necessary if the anti-virus moiety were to be directly chemically crosslinked with the matrix. Direct chemical cross-linking between an anti-virus moiety and the matrix, in the absence of a matrix-binding domain, is not desirable because the conditions required for the cross-linking reaction would have negative impact on the function of antibody.

Hence, in one embodiment of the invention, the anti-virus moiety is coupled to a matrix-binding domain, wherein the anti-virus moiety is immobilised in the matrix via the interaction between the matrix-binding domain and the matrix. The anti-virus moiety is not immobilised in a matrix by chemical crosslinking between the anti-virus moiety and the matrix.

The matrix may be a readily available resource such as cotton or polyester fabric, paper, or polyester fibre. Matrices from such readily available resources can easily be obtained (e.g. from retail outlets) and the anti-virus moiety may be immobilised in the matrix via the interaction between the matrix-binding domain and the matrix. Immobilisation may be performed easily and anywhere, e.g. at home. Hence, a science laboratory is not necessary for immobilisation to be performed. This is particularly the case when the fusion protein of the invention is provided in solution.

Further advantages of using polyester as the binding matrix include easy removal of the anti-virus moiety and bound virus, e.g. using household items, such as detergent or hot water treatment, as described herein.

The matrix, in particular, polyester, can be reused once washed, as described herein.

### Fusion Protein

The invention also provides a fusion protein comprising: (i) an anti-virus moiety, such as an anti-virus moiety described herein, (ii) a matrix-binding domain, and/or (iii) a linker between the anti-virus moiety and the matrix-binding domain. The fusion protein may comprise: (i) an anti-virus moiety, such as an anti-virus moiety described herein, and (ii) a matrix-binding domain. The fusion protein may comprise: (i) an anti-virus moiety described herein, (ii) a matrix-binding domain, and (iii) a linker between the anti-virus moiety and the matrix-binding domain.

The fusion protein may further comprise a solubility tag and/or an affinity tag, as described above.

The anti-virus moiety may specifically bind to an epitope in a coronavirus, such as SARS-COV-2. The anti-virus moiety may specifically bind to an epitope in a virus (e.g. a coronavirus, such as SARS-COV-2) with a K_{D} value of between 1 to 10 nM. The anti-virus moiety may comprise an amino acid sequence as described herein, e.g. having ≥70% sequence identity with any one of SEQ ID NOs: 1 to 3.

The anti-virus moiety may comprise an amino acid sequence as described herein, e.g. having >70% sequence identity with any one of SEQ ID NOs: 46 to 48 and 82 to 84.

The matrix-binding domain and the linker between the anti-virus moiety and the matrix-binding domain may be the same protein, e.g. functionalised spider silk.

the anti-virus moiety is an antibody, optionally wherein the antibody is a single chain variable fragment (scFv).

“Fusion proteins” as employed herein comprise a protein component fused to one or more protein component.

The fusion protein is a protein expressed by recombinant techniques from a genetic construct, for example expressed in a host from a DNA construct.

The fusion protein may be expressed as a “single protein/unit” from a cell. In the case of a fusion protein comprising a Fab/Fab′ fragment there will be two chains but this will be considered a single protein for the purpose of the present specification with one chain.

For example, a fusion protein of the invention comprising: (i) an anti-virus moiety, such as an anti-virus moiety described herein, (ii) a matrix-binding domain, and (iii) a linker between the anti-virus moiety and the matrix-binding domain may be expressed as a “single protein/unit” from a cell.

Alternatively, the fusion protein may be expressed separately from a cell and subsequently fused together, e.g. through spytag technology. For example, a fusion protein of the invention comprising: (i) an anti-virus moiety, such as an anti-virus moiety described herein, (ii) a matrix-binding domain, and (iii) a linker between the anti-virus moiety and the matrix-binding domain may be expressed from separate cells, and subsequently fused together, e.g. through spytag technology.

The fusion protein of the invention, when immobilised in a matrix, has good stability, as shown in the Examples. For example, the fusion protein may be stored at 4° C., room temperature or 37° C. for an extended period of time, e.g. for 1, 2, 3, 4, 5 or 6 weeks.

### Linker

The peptides discussed herein may comprise a linker. The linker allows the peptides to retain biological function by creating a physical distance and/or separation.

The teaching herein of linkers in one context can equally be applied to linkers in different contexts where a linker is employed, such as in a particular anti-virus moiety or a matrix-binding domain described herein. For example, a linker may be used to join the light and heavy chains of the antigen-binding region in a scFv. A linker may be used to join the various components of a fusion protein of the invention, e.g. between the anti-virus moiety and a matrix-binding domain or between the anti-virus moiety and a solubility tag.

Examples of linkers useful with the invention are known in the art, e.g. the ones used in the Examples below. Useful linkers include, for example, recombinant spider silk protein, elastin-like polypeptide, troponin C fragment and/or GRG₅RG₄R (GB1-resilin polymer).

In some embodiments, a protein may have dual characteristics, e.g. functionalised spider silk, which may be the linker and the matrix-binding domain.

### Applications and Hygiene Products

The invention relates to using the anti-virus moiety immobilised in a matrix to prevent virus (e.g. SARS-COV-2) transmission, e.g. by filtering or removing virus particles and/or fragments thereof (e.g. SARS-COV-2) from the environment or a surface.

The immobilised anti-virus moiety may filter or remove ≥10% (i.e. 10% or more), ≥20%, ≥30%, ≥40%, ≥50%, ≥60%, ≥70%, ≥80%, ≥90% or 100% of the virus particles and/or fragments thereof from the environment or a surface.

The immobilised anti-virus moiety may be in contact with the environment or a surface, e.g. soil, water or air, for a time and under conditions sufficient for a target virus particle to be retained by the immobilized anti-virus moiety. The retained virus particles may be analysed in situ or removed (e.g. eluted) for further analysis, e.g. biochemically, immunologically or microscopically. Alternatively, the hygiene product containing retained virus particles may simply be discarded.

The matrix of the invention is suitable for use as a hygiene product. For example, the matrix of the invention may be part of the hygiene product. Hence, a hygiene product of the invention may be prepared by incorporating a matrix of the invention.

One of the advantages of the invention is that the matrices described herein can be incorporated in a hygiene product after drying (e.g. air-dried) and storage. Hence, the hygiene product may be a wet or dry product. As demonstrated in the Examples, the anti-virus moiety and fusion proteins of the invention, when immobilized in matrix and then dried, remain active and functional.

In embodiments where the hygiene product is a liquid, the immobilised anti-virus moiety may be at a concentration of between 1 ng/ml and 1 mg/ml (i.e. between 1.63 BVB (billion virus copy binding units) and 1.63×10⁶ BVB).

In embodiments where the hygiene product is a solid surface, the immobilised anti-virus moiety may be at a concentration of between 1 ng/cm² to 1 mg/cm² (i.e. between 1.63 BVB (billion virus copy binding units) and 1.63×10⁶ BVB). For example, in embodiments where the hygiene product is a bead, the immobilised anti-virus moiety may be at a concentration of between 1 ng/ml and 1 mg/ml (i.e. between 1.63 BVB (billion virus copy binding units) and 1.63×10⁶ BVB).

The hygiene product may be a personal protective equipment, such as lab coat, protective mask (e.g. face mask, mask, respirator, face shield, surgical mask, medical mask, filter mask, mouth mask, or gas mask), shoe cover, or hair cap. The personal protective equipment may be in the form of a clothing item, such as an everyday clothing item, e.g. a hijab or a veil.

The hygiene product may be a filter, such as an air filter (e.g. HEPA filter) or a water filter.

The hygiene product may comprise a polyethylene membrane.

The hygiene product may be a household product. The household product may be in the form of a sheet, such as tissue, surface wipe, linen, napkin, curtain, or table cloth. The household product may be in the form of a liquid or semi-liquid, such as cleaning solution (e.g. aerosol spray), sanitiser gel or cream. The household product may be an infant product, such as diaper, wipe and toy.

The solution may be a cellulose solution.

The matrix of the invention is also suitable for use as a clothing item. Such a clothing item may prevent virus transmission, e.g. by filtering or removing virus particles and/or fragments thereof from, e.g. the environment. Hence, such a clothing item may have the additional purpose as a hygiene product. For example, the matrix of the invention may be part of the clothing item. Hence, a clothing item of the invention may be prepared by incorporating a matrix of the invention. The clothing item may be a hijab or a veil.

One of the advantages of the invention is that the matrices described herein can be incorporated in a clothing item after drying (e.g. air-dried) and storage. As demonstrated in the Examples, the anti-virus moiety and fusion proteins of the invention, when immobilized in matrix and then dried, remain active and functional.

In embodiments where the anti-virus moiety is immobilised in a matrix in a reversible manner, the hygiene product or the clothing item comprising such anti-virus moiety is reusable. For example, the anti-virus moiety may be removed from a hygiene product or a clothing item by washing, e.g. with a detergent such as a surfactant (e.g. Sodium lauroyl sarcosinate, Triton X-100 and Tween-20), with acidic solution (e.g. acidic methanol (e.g. HOAc/MeOH)) or with water, preferably at high temperature (e.g. at 100° C.), as shown in the Examples. New anti-virus moiety may then be immobilised onto the hygiene product or clothing item.

The invention relates to a method of preventing virus transmission, e.g. by filtering or removing virus particles and/or fragments thereof (e.g. SARS-COV-2) from the environment or a surface, using the immobilised anti-virus moiety of the invention. Similarly, the invention also relates to the use of the immobilised anti-virus moiety of the invention to prevent virus transmission, e.g. to filter or remove virus particles and/or fragments thereof (e.g. SARS-COV-2) from the environment or a surface.

The invention relates to preventing virus transmission in the air.

The immobilised anti-virus moiety of the invention may be used in filters and protective masks to prevent entry of the target virus (e.g. SARS-COV-2) into the respiratory tract. For example, the protective mask may be worn by a subject (e.g. who is not infected by coronavirus (e.g. SARS-COV-2)) to protect himself from coronavirus (e.g. SARS-COV-2) infection.

The immobilised anti-virus moiety of the invention may be used in filters and protective masks to prevent the spread of the target virus (e.g. SARS-COV-2) into the environment or surfaces from a subject infected by coronavirus (e.g. SARS-COV-2). For example, the protective mask may be worn by a subject who is infected by coronavirus (e.g. SARS-COV-2) and is wearing the face mask to prevent the spread of coronavirus (e.g. SARS-COV-2) into the environment or surfaces.

Alternatively, the immobilised anti-virus moiety of the invention may be used in aerosol sprays to bind the respiratory droplets that contain the target virus (e.g. SARS-CoV-2).

The method or use of the invention may comprise filtering the air through a filter of the invention.

The method or use of the invention may comprise a subject wearing a personal protective equipment of the invention, e.g. a protective mask.

The method or use of the invention may comprise a subject breathing though a protective mask of the invention.

The invention relates to preventing virus transmission in the water. The immobilised anti-virus moiety of the invention may be used in a water filter. The method or use of the invention may comprise filtering water through a filter of the invention.

The invention relates to preventing fomite transmission. The immobilised anti-virus moiety of the invention may be used in a cleaning product, e.g. cleaning solution (e.g. aerosol spray), sanitiser gel or cream, to remove the target virus from a surface.

The method or use of the invention may comprise spraying or wiping a hard surface with a cleaning solution (e.g. aerosol spray).

The method or use may comprise rubbing sanitiser gel or cream to a body part, e.g. hands.

Typically, the invention relates to methods and uses for a human subject in need thereof. However, non-human animals such as rats, rabbits, sheep, pigs, cows, cats, or dogs is also contemplated.

The subject may be anyone in the population.

The subject may be at risk of exposure to coronavirus infection, such as a healthcare worker or a person who has come into contact with an infected individual. A subject may have visited or be planning to visit a country known or suspected of having a coronavirus outbreak. A subject may also be at greater risk, such as an immunocompromised individual, for example an individual receiving immunosuppressive therapy or an individual suffering from human immunodeficiency syndrome (HIV) or acquired immune deficiency syndrome (AIDS).

The subject may be asymptomatic or pre-symptomatic.

The subject may be early, middle or late phase of the disease.

The subject may be in hospital or in the community at first presentation, and/or later times in hospital.

The subject may be male or female. In certain embodiments, the subject is typically male.

The subject may not have been infected with coronavirus, such as SARS-COV-2.

The subject may have a predisposition to the more severe symptoms or complications associated with coronavirus infections. The method or use of the invention may comprise a step of identifying whether or not a patient is at risk of developing the more severe symptoms or complications associated with coronavirus.

The subject may or may not have been diagnosed to be infected with coronavirus, such as SARS-COV-2.

### Composition or Combination

A matrix or a hygiene product of the invention may comprise multiple anti-virus moieties, wherein each anti-virus moiety is specific for a different epitope. The different epitopes may be in the same virus or may be in different viruses.

Hence, the invention also provides a composition comprising multiple anti-virus moieties, immobilised in the same or different matrices of the invention. The invention also provides a hygiene product comprising such a composition.

The immobilised anti-virus moiety of the invention may be used in combination with one or more biocides. A biocide may be chlorine dioxide, hydrogen peroxide, peroxy acid, an alcoholic or a phenolic compound, an essential oil and its effective component, bleach, an antibiotic, or an antimicrobial phytochemical. Useful essential oils may include thyme oil, tea tree oil, rosemary oil, eucalyptus oil, or citral oil.

Hence, a matrix or a hygiene product of the invention may comprise the immobilised anti-virus moiety of the invention and a biocide.

A method or use of the invention may comprise applying the immobilised anti-virus moiety of the invention in combination with a biocide to prevent virus transmission (e.g. SARS-COV-2), e.g. by filtering or removing virus particles and/or fragments thereof from the environment or a surface.

### Other

It is to be understood that different applications of the disclosed immobilised anti-virus moiety, fusion protein and/or matrix of the invention may be tailored to the specific needs in the art. It is also to be understood that the terminology used herein is for the purpose of describing particular embodiments of the invention only, and is not intended to be limiting.

In addition, as used in this specification and the appended claims, the singular forms “a”, “an”, and “the” include plural references unless the content clearly dictates otherwise. Thus, for example, reference to “an antibody” includes two or more antibodies.

Furthermore, when referring to “≥x” herein, this means equal to or greater than x. When referring to “≤y” herein, this means equal to or less than y.

For the purpose of this invention, in order to determine the percent identity of two sequences (such as two polynucleotide or two polypeptide sequences), the sequences are aligned for optimal comparison purposes (e.g. gaps can be introduced in a first sequence for optimal alignment with a second sequence). The nucleotides at each position are then compared. When a position in the first sequence is occupied by the same nucleotide as the corresponding position in the second sequence, then the nucleotides are identical at that position. The percent identity between the two sequences is a function of the number of identical positions shared by the sequences (i.e., % identity=number of identical positions/total number of positions in the reference sequence×100).

Typically the sequence comparison is carried out over the length of the reference sequence. For example, if the user wished to determine whether a given (“test”) sequence is 95% identical to SEQ ID NO: 3, SEQ ID NO: 3 would be the reference sequence. To assess whether a sequence is at least 95% identical to SEQ ID NO: 3 (an example of a reference sequence), the skilled person would carry out an alignment over the length of SEQ ID NO: 3, and identify how many positions in the test sequence were identical to those of SEQ ID NO: 3. If at least 95% of the positions are identical, the test sequence is at least 95% identical to SEQ ID NO: 3. If the sequence is shorter than SEQ ID NO: 3, the gaps or missing positions should be considered to be non-identical positions.

The skilled person is aware of different computer programs that are available to determine the homology or identity between two sequences. For instance, a comparison of sequences and determination of percent identity between two sequences can be accomplished using a mathematical algorithm. In an embodiment, the percent identity between two amino acid or nucleic acid sequences is determined using the Needleman and Wunsch (1970) algorithm which has been incorporated into the GAP program in the Accelrys GCG software package (available at http://www.accelrys.com/products/gcg/), using either a Blosum 62 matrix or a PAM250 matrix, and a gap weight of 16, 14, 12, 10, 8, 6, or 4 and a length weight of 1, 2, 3, 4, 5, or 6.

All publications, patents and patent applications cited herein, whether supra or infra, are hereby incorporated by reference in their entirety.

The following examples illustrate the invention.

### Example 1

This experiment aims to express and purify anti-virus moieties, each coupled to a cellulose-binding domain, also referred to herein as Karunyam proteins. The binding properties of each of the Karunyam proteins are also investigated.

### Materials and Methods

### DNA Constructs

Three Karunyam proteins, each containing a scFv coupled to a matrix-binding domain (cellulose binding motif 3, CBM3), were prepared. scFv sequences were synthesised from a commercial source (Genscript) with the format VH-linker-VL, based on available published sequences of monoclonal antibodies P2B2F6, P2C1F11 and P2C1A3 as disclosed in References 5 and 6. A parent vector was created using a cold-induction vector (pCOLD-I), with an N-terminal histidine tag and glutathione-S-transferase solubility enhancement tag. At the C-terminal, a cellulose-binding motif (CBM3) was introduced. scFv constructs were cloned in frame into the parent vector to create the bacterial expression vectors. The resulting Karunyam proteins have the sequences set out in Table 2.

TABLE 2
Features of the Karunyam proteins.

| | | | Matrix binding |
|---|---|---|---|
| Karunyam | Anti-virus | | protein at |
| protein | moiety | N-terminal tag | C terminus |
| Karunyam 9 | scFv 9 | His-tag | CBM3 (SEQ |
| (SEQ ID NO: 7) | (SEQ ID NO: 1) | (SEQ ID NO: 4) | ID NO: 6) |
| Karunyam 10 | scFv 10 | and | |
| (SEQ ID NO: 8) | (SEQ ID NO: 2) | GST-tag | |
| Karunyam 11 | scFv 11 | (SEQ ID NO: 5) | |
| (SEQ ID NO: 9) | (SEQ ID NO: 3) | | |

### Protein Expression and Affinity Purification

DNA constructs were expressed in the BL21 (DE3) strain of E. coli. An overnight culture of bacterial culture was plated to create a confluent plate. Half of the cells from a confluent plate were scraped off and resuspended in 100 mL of 2×YT broth. After growth at 37° C. for 3 hours, the cells were cooled on ice for 30 minutes, IPTG was added to 0.5 mM, and the cell were allowed to incubate with shaking at 15° C. for 21 hours. The cells were centrifuged into a pellet, then resuspended in lysis buffer (8 mL PBS, 2 mL PopCulture reagent, 2 μL Lyonase). The resuspended cells were disrupted using three freeze-thaw cycles using liquid nitrogen. A combination of GST magnetic beads and Nickel-NTA magnetic beads (250 μL each) were added to the culture, with stirring at room temperature for 30 minutes. The magnetic beads were denatured overnight at 4° C. in a mild denaturing solution (2M urea, 1 mM DTT, 0.05% Tween 20). The next day, the refolding process was carried out with two successive 20 minute incubations with 1M and 0.5M urea solutions containing 1 mM DTT and 0.05% Tween 20 as well. The protein was eluted first with an elution buffer containing 15 mM Glutathione, then with a second elution buffer containing 500 mM imidazole.

To improve the expression and solubility of the scFvs, the following reagents and conditions were tested: choice of cell strain, choice of N-terminal solubility tags, optimization of protein induction time, optimization of IPTG concentration during induction, and use of appropriate solubilization and denaturation/refolding buffers. The results are summarised below:
1. Use of different E. Coli strains to optimise expression: BL21 (DE3) LysS, BL21 (DE3), DRX, and NovaBlue were used. The best expression strain was found to be BL21 (DE3) which was used for further experiments.
2. Solubility tags were attached to the N-terminus of the fusion proteins, including GB1, GST, SUMO, MBP and NusA. The GST fusion proteins showed greatest solubility and ease of purification and were used for further experiments.
3. Induction time at 15 degrees Celsius was optimised from 6 hours to 21 hours. The final experimental conditions involved 3 hours of cell growth at 37 degrees, followed by induction at 15 degrees for 21 hours.
4. IPTG concentration was optimized to 0.5 mM during the protein induction phase at 15 degrees Celsius.
5. Solubilization and denaturation/renaturation were carried out using a variety of buffers. The best yield was obtained using the conditions described above.

### Far-Western Blot Analysis

Affinity purified Karunyam proteins were immobilised on nitrocellulose (NC) membranes using a slot-blot apparatus. Serial dilutions of a control monoclonal antibody to the RBD of the spike protein of SARS-COV-2 (hCoV-19/Wuhan/WIV04/2019 (WIV04) (GISAID accession no. EPI_ISL_402124) was added to 4 wells as a positive control. The NC blot was dried for one hour, blocked with 5% milk in TBST, and treated overnight at 4° C. with a solution of RBD-spike protein conjugated to HRP (Genscript, catalogue L00847; amino acids 319-Ser591). The next day, the blot was washed three times with TBST, then developed using ECL imaging.

### Enzyme-Linked Immunosorbent Assay (ELISA)

Affinity-purified Karunyam proteins were coated overnight on glutathione-lined 96-well ELISA plates (200 ng/100 μL) at 4° C. The next day, plates were washed twice with Tris-buffered Saline pH 8.0 containing 0.1% Tween-20 (TBST), then allowed to incubate with serial dilutions of biotinylated RBD-spike protein/Streptavidin-HRP mixture for 1 hour at 37° C. The biotinylated RBD spike was from BIOSSUSA, amino acids Arg319-Ans532. Plates were washed twice with TBST. 3,3′,5,5′-tetramethylbenzidine (TMB, Fisher) solution was added for 15 minutes at room temperature, and then Stop solution (0.16M sulfuric acid, Fisher) was added to quench the enzyme reaction. Plates were read at 450 nM using a Cytation 5 image reader.

### Results

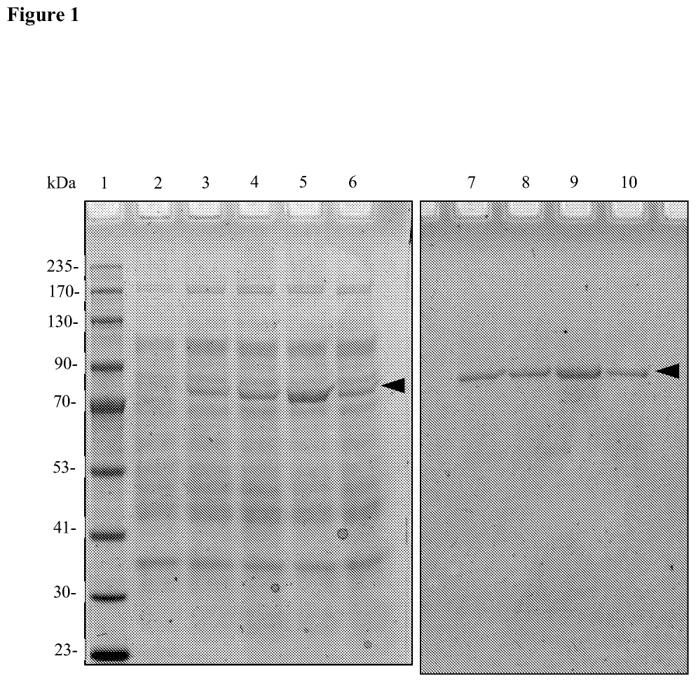

Vectors containing sequences encoding the Karunyam proteins were prepared in bacterial cultures and vector expression was induced. Bacterial cultures were then lysed by freeze-thaw cycles. Karunyam proteins were unfolded and refolded using denaturants, and then affinity purified using nickel magnetic beads and glutathione magnetic beads. As shown in FIG. 1, four preparations of Karunyam 9 were affinity purified and analysed by 4-12% SDS-PAGE. Lane 1 shows the molecular weight markers in kDa. Lane 2 shows a sample of pre-induction cell lysate. Lanes 3-6 show cell lysates after vector expression was induced by cold induction. Lanes 7-10 show the affinity purified protein. The black arrows indicate the band that corresponds to the size of the Karunyam 9 protein. The gel in FIG. 1 therefore demonstrates that Karunyam 9 can be expressed from bacteria and that subsequent affinity purification successfully enriches Karunyam 9 from cell lysate.

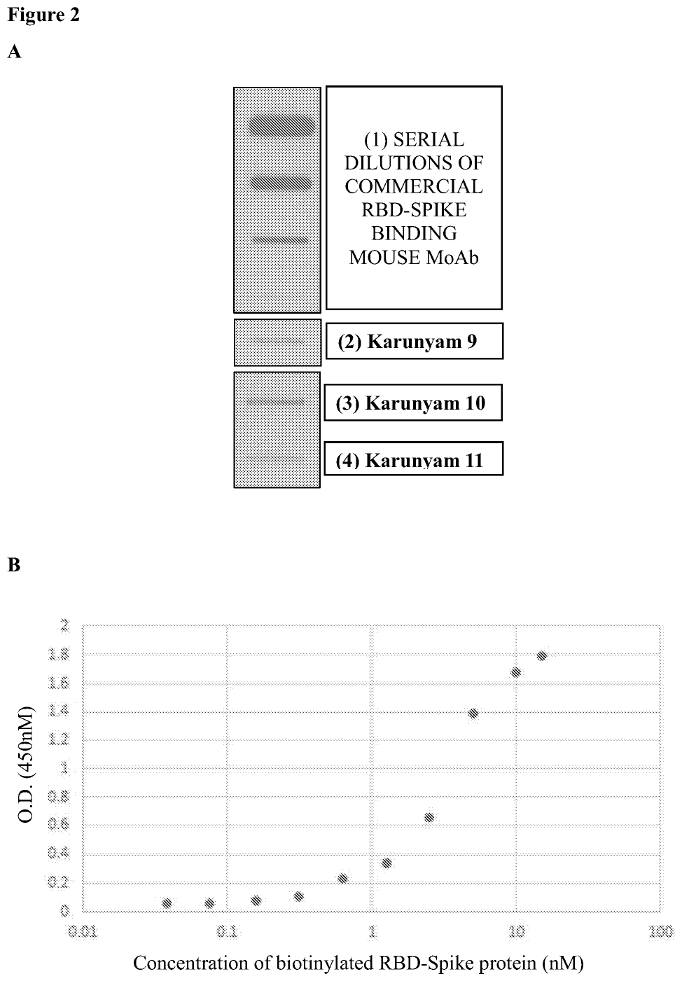

To test whether the Karunyam proteins could bind to the RBD-spike protein, the affinity purified Karunyam proteins were immobilised on a nitrocellulose membrane and RBD-spike protein conjugated to HRP was added. Detection of signal during ECL imaging is indicative of specific binding of the RBD-spike protein to the Karunyam protein. Results of the Far-Western blot assay are shown in FIG. 2A. Three distinct bands were detected on the blot, corresponding to Karunyam 9, Karunyam 10 and Karunyam 11. Accordingly, Karunyam 9, Karunyam 10 and Karunyam 11, when immobilised on a membrane, were all capable of binding to the RBD-spike protein.

To quantify the binding affinity of the Karunyam proteins to the RBD-spike protein, an ELISA was performed according to the method described above. FIG. 2B shows a dose response curve for Karunyam 9 with values of optical density at 450 nM against increasing concentration of RBD-spike protein. The K_{D} for each Karunyam protein was estimated from dose response curves, and is shown in Table 3 below. For comparison, Table 4 shows the K_{D} for the known antibodies from which the scFvs were derived, and the K_{D} for the human Angiotensin-converting enzyme 2 (ACE2) receptor which naturally binds to the RBD. As shown in Table 3, each of Karunyam 9, Karunyam 10 and Karunyam 11 binds to the RBD-spike protein with high affinity. The binding affinity of these proteins for the RBD-spike protein is similar to that of the human ACE2 receptor for the RBD-spike protein of SARS-COV-2, and to that of the known antibodies on which they are based.

TABLE 3
Binding affinity of Karunyam proteins at 37° C.

| Karunyam | Anti-virus moiety | K_{D} (nM) |
|---|---|---|
| Karunyam 9 | scFv 9 | 4.05 ± 0.26 |
| Karunyam 10 | scFv 10 | 6.35 ± 0.65 |
| Karunyam 11 | scFv 11 | 7.88 ± 2.06 |

TABLE 4
Binding affinity of known antibodies of the prior art at 37° C.

| | Parent | | |
|---|---|---|---|
| Anti-virus moiety | antibody | K_{D} (nM) | Reference |
| scFv 9 | P2B-2F6 | 5.14 | 6 |
| scFv 10 | P2C-1F11 | 2.12 | 6 |
| scFv 11 | P2C-1A3 | 2.47 | 6 |
| ACE2 receptor | N/A | 4.7 to SARS-CoV2-RBD | 5 |
| | | 31 to SARS-CoV-RBD | |

### Example 2

This experiment aims to elucidate whether the Karunyam proteins, when immobilised on cellulose beads, can bind to viral pseudoparticles in solution. Viral pseudoparticles consist of the RBD-spike protein conjugated to FITC nanoparticles, which have a diameter of approximately 100 nm. Coronavirus particles have a diameter of approximately 60-140 nm, so these viral pseudoparticles act as a model for the coronavirus particles.

### Materials and Methods

### Cellulose Bead Binding Assay

Cellulose beads of 1 mm average diameter were coated overnight with purified Karunyam protein at 4° C., using 200 ng of Karunyam protein in a total volume of 1 mL PBS in each well of a 12 well culture plate. One in two serial dilutions were prepared of RBD-spike protein conjugated to FITC nanoparticles (viral pseudoparticles). 60 μL of each dilution was estimated for RBD-spike protein using immunochromatography (IC) strips. The reactivity was estimated after 15 minutes. Beads were washed twice with TBST and then treated for one hour at 37° C. with serial dilutions of RBD-spike protein conjugated with FITC nanoparticles. The unbound RBD-spike protein content was estimated using IC strips.

For RBD-spike protein content, RBD spike protein dilutions in 100 μL TBST were prepared ranging from 20 nM to 0.15 nM. 60 μL of each dilution was spotted on the IC strip and allowed to develop over 15 minutes. The presence of the positive band indicates the presence of RBD-spike protein. The threshold sensitivity of the IC strip was determined under these conditions as 0.63 nM.

The cellulose beads with immobilised Karunyam protein were added to the serial dilutions of RBD-spike protein. The RBD-spike protein dilutions were then re-tested using the IC strips to estimate the amount of unbound RBD-spike protein after treatment with the beads. Table 5 shows the serial dilutions of RBD-spike protein and the qualitative IC strip results before and after treatment with immobilised Karunyam proteins. The difference in IC testing between pre- and post-Karunyam treatment RBD-spike protein provided the approximate amounts of RBD-spike protein bound to immobilised Karunyam proteins.

### Results

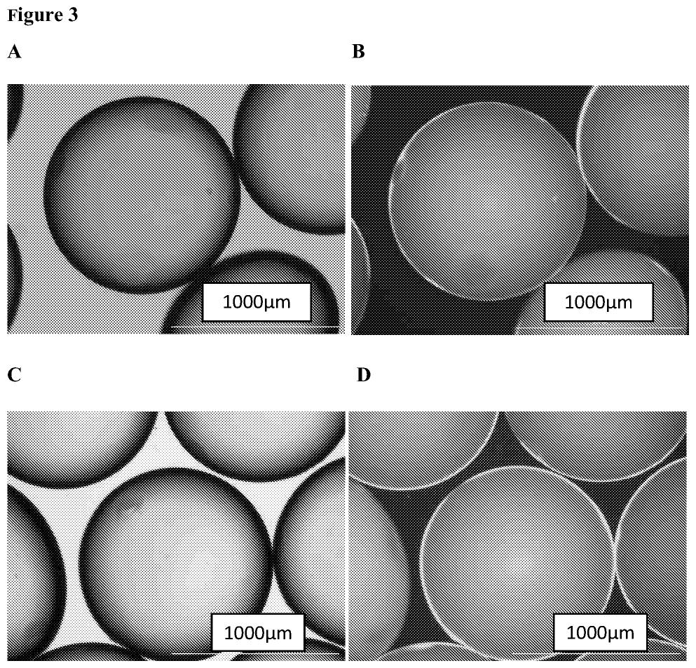

To test whether the Karunyam proteins when bound to a cellulose matrix can bind to virus pseudoparticles, cellulose bead binding assays were performed. Karunyam proteins immobilised on cellulose beads were treated with serial dilutions of RBD-spike protein conjugated with FITC nanoparticles. The FITC nanoparticles have a diameter of 100 nm so are representative of coronavirus particles, which are 60-140 nm in diameter. As shown in FIGS. 3A and B, fluorescence of the cellulose beads was detected after 5 minutes, demonstrating that the Karunyam proteins bound to the cellulose beads can bind the RBD-spike protein conjugated with FITC nanoparticles. When left in solution for one week, the Karunyam proteins bound to the cellulose beads continued to bind the RBD-spike protein, as demonstrated in FIGS. 3C and D.

To estimate the amount of RBD-spike protein bound to the Karunyam proteins immobilised on the cellulose beads, IC strips were used to firstly estimate the amount of total RBD-spike protein in the serial dilutions, and then to estimate the amount of unbound RBD-spike protein after addition to the beads. The difference between total and unbound RBD-spike protein provided the approximate amounts of RBD-spike protein bound to immobilised Karunyam proteins, as shown in Table 5.

TABLE 5
Estimated amount of RBD-spike protein bound to Karunyam
proteins immobilized on cellulose beads after 15 minutes.

| | Dilution of RBD-spike protein | | | | | | |
|---|---|---|---|---|---|---|---|
| | 10 nM | 5 nM | 2.5 nM | 1.25 nM | 0.63 nM | 0.31 nM | 0.15 nM |
| TOTAL | + | + | + | + | + (0.63) | − | − |
| Karunyam 9-unbound | + | + (0.63) | − | − | − | − | − |
| Karunyam 10-unbound | + | + (0.63) | − | − | − | − | − |
| Karunyam 11-unbound | + | + (0.63) | − | − | − | − | − |

The + sign indicates the presence of a positive signal on the IC strip.
The − sign indicates absence of a signal on the IC strip.

### Example 3

This experiment investigates the extent to which the Karunyam proteins, when immobilised on cellulose beads, can retain virus pseudoparticles.

Karunyam proteins bound to cellulose beads were subject to the virus pseudoparticle trap assay. In detail, cellulose beads of 1 mm average diameter were coated overnight with Karunyam protein at 4° C., using 200 ng of Karunyam protein in a total volume of 0.5 mL PBS in a spin-column. RBD-spike protein conjugated to FITC nanoparticles (virus pseudoparticles) at a concentration of 10 nM were incubated in each spin column in a volume of 200 μL for 30 minutes at 37° C. The spin columns were centrifuged at 14,000 g for 5 minutes. The unbound RBD-spike protein fraction was determined using a competitive ELISA. The difference between total and unbound RBD-spike protein as provided by IC analysis provides a value of the RBD-spike protein bound to cellulose beads.

For competitive ELISA, affinity-purified SCFVs were coated overnight on Glutathione plates (200 ng/100 ul) at 4° C. The next day, plates were washed twice with Tris-buffered Saline pH 8.0 containing 0.1% Tween-20 (TBST), then allowed to incubate with serial dilutions of biotinylated RBD-Spike protein/Streptavidin-HRP mixture for 1 hour at 37° C., in the presence/absence of competitor (Total Spike-RBD/FITC added to spin-column (Total), or Unbound Spike-RBD/FITC eluate from the spin-column (Unbound)).

Plates were washed twice with TBST. 3,3′,5,5′-tetramethylbenzidine (TMB, Fisher) solution was added for 15 minutes at room temperature, and then Stop solution (0.16M sulfuric acid, Fisher)) was added to quench the enzyme reaction. Plates were read at 450 nM using a Cytation 5 image reader. The amount of unbound Spike-RBD/FITC was determined as the difference between the Total-Unbound.

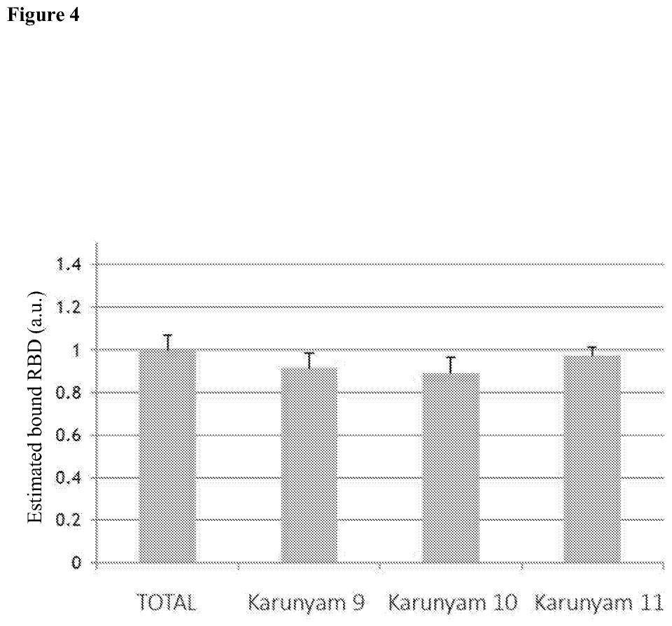

The results of the pseudoparticle trap assay are shown in FIG. 4. Karunyam 9, Karunyam 10 and Karunyam 11 all retained a large proportion of the total virus pseudoparticles after centrifugation. On average, the percentage of total virus pseudoparticles that are retained by the Karunyam proteins is 93%.

These results show that Karunyam 9, Karunyam 10 and Karunyam 11 immobilised on cellulose beads are able to retain virus pseudoparticles after centrifugation. Accordingly, these proteins can act as “traps” for virus particles and/or fragments thereof.

### Example 4

Since one Karunyam molecule can bind only one RBD-spike protein, there is a 1:1 ratio as a theoretical maximum for binding. Accordingly this Example aims to calculate the approximate number of viral copies that would be bound by a specified amount of Karunyam protein.

As demonstrated in Example 3, the percentage of total virus pseudoparticles is 93%. As shown in FIG. 4, the percentage of total virus pseudoparticles retained by immobilised Karunyam 9 is approximately 90%. 200 ng Karunyam 9 is able to bind to 90% virus pseudoparticles in the solution i.e. 90% of 30 ng RBD-spike protein, which is approximately 27 ng RBD-spike protein. The approximate molecular weight of the RBD-spike protein is 50 kDa. Accordingly, 1 ng Karunyam 9 can bind 1.625×10⁹ viral copies.

According to Reference 9, patients recovering from SARS-COV-2 exhale approximately 7×10⁴ viral copies per hour. Karunyam 9 is immobilised in a face mask at a concentration of about 1 ng/cm³. The face mask is used by a patient recovering from COVID19. The face mask is capable of sequestering SARS-COV-2 exhaled from the recovering patient for approximately 23,000 hours, which is approximately 2.5 years.

According to Reference 10, patients who are suffering from active SARS-COV-2 infections exhale approximately 2.94×10⁴ viral copies per minute, which is approximately 1.76×10⁶ copies per hour. Karunyam 9 is immobilised in a face mask at a concentration of about 1 ng/cm³. The face mask is used by a COVID19 patient. The face mask is capable of sequestering SARS-COV-2 exhaled from the patient for approximately 900 hours, which is approximately 5 weeks.

The face mask is worn by a COVID19 patient in hospitals and enclosed places, such as aircraft, to prevent SARS-COV-2 transmission and protect others from SARS-COV-2 infections.

### Example 5-SARS-COV-2 Strains

This experiment aimed to express and purify Karunyam proteins that target Delta and Omicron variants of SARS-COV-2. The binding properties of each of the Karunyam proteins were also investigated. This experiment also investigated the extent to which the Karunyam proteins, when immobilised on cellulose beads, can retain virus pseudoparticles.

### Materials and Methods

### DNA Constructs

Six Karunyam proteins, each containing a scFv coupled to a matrix-binding domain (cellulose-binding motif 3, CBM3), were prepared. scFv sequences were synthesised from a commercial source (Genscript) with the format VH-linker-VL, based on available published sequences of monoclonal antibodies SARS 2-38, CT-P59, LY-COV1404, S2X259, S2H97, and S2K146 as disclosed in References 11, 12, 13, 14. scFv constructs were cloned according to the methods described in Example 1 to create bacterial expression vectors. The resulting Karunyam proteins have the sequences set out in Table 6 and Table 7.

TABLE 6
Features of the Karunyam proteins that target the Delta spike protein.

| | | | Matrix binding |
|---|---|---|---|
| Karunyam protein | Anti-virus moiety | N-terminal tag | protein at C terminus |
| Karunyam 13 | scFv 13 | His-tag | CBM3 (SEQ ID NO: 6) |
| (SEQ ID NO: 49) | (SEQ ID NO: 46) | (SEQ ID NO: 4) | |
| Karunyam 14 | scFv 14 | and | |
| (SEQ ID NO: 50) | (SEQ ID NO: 47) | GST-tag | |
| Karunyam 15 | scFv 15 | (SEQ ID NO: 5) | |
| (SEQ ID NO: 51) | (SEQ ID NO: 48) | | |

TABLE 7
Features of the Karunyam proteins that target the Omicron spike protein.

| | | | Matrix binding |
|---|---|---|---|
| Karunyam protein | Anti-virus moiety | N-terminal tag | protein at C terminus |
| Karunyam 18 | scFv 18 | His-tag | CBM3 (SEQ ID NO: 6) |
| (SEQ ID NO: 85) | (SEQ ID NO: 82) | (SEQ ID NO: 4) | |
| Karunyam 19 | scFv 19 | and | |
| (SEQ ID NO: 86) | (SEQ ID NO: 83) | GST-tag | |
| Karunyam 20 | scFv 20 | (SEQ ID NO: 5) | |
| (SEQ ID NO: 87) | (SEQ ID NO: 84) | | |

### Protein Expression and Affinity Purification

Karunyam proteins were expressed and affinity purified according to the methods described in Example 1.

### Enzyme-Linked Immunosorbent Assay (ELISA)

ELISA experiments were performed according to the method described in Example 1, except a biotinylated Delta RBD-spike protein from Sinobiological, amino acids Arg319-Phe541, was used in experiments using the Karunyam proteins that target the Delta variant of SARS-COV-2 (Karunyam proteins 13, 14 and 15), and a biotinylated Omicron RBD-spike protein from Sinobiological, amino acids Arg319-Phe541, was used in experiments using the Karunyam proteins that target the Omicron variant of SARS-CoV-2 (Karunyam proteins 18, 19 and 20).

### Cellulose Bead Binding Assay

Cellulose bead binding assays were performed according to the method described in Example 2, except the Delta RBD-spike protein or the Delta-plus RBD-spike protein was conjugated to FITC nanoparticles (viral pseudoparticles) in assays that detect binding to Karunyam proteins targeting the Delta variant of SARS-COV-2 (Karunyam proteins 13, 14 and 15), and the Omicron RBD-spike protein was conjugated to FITC nanoparticles in assays that detect binding to Karunyam proteins targeting the Omicron variant of SARS-CoV-2 (Karunyam proteins 18, 19 and 20).

### Results

To quantify the binding affinity of the Karunyam proteins to the RBD-spike protein from the Delta variant of SARS-COV-2 and to the RBD-spike protein from the Omicron variant of SARS-COV-2, ELISA experiments were performed according to the method described above. K_{D} value for each Karunyam protein was estimated from dose response curves, and are shown in Table 8 below. For comparison, Table 9 shows the K_{D} values for the known antibodies from which the scFvs were derived, and the K_{D} for the human ACE2 receptor which naturally binds to the RBD. As shown in Table 8, each of Karunyam 13, Karunyam 14 and Karunyam 15 binds to the Delta RBD-spike protein with high affinity and each of Karunyam 18, Karunyam 19 and Karunyam 20 binds to the Omicron RBD-spike protein with high affinity. The binding affinity of these proteins for the Delta RBD-spike protein is similar to that of the human ACE2 receptor for the RBD-spike protein of SARS-COV-2, and to that of the known antibodies on which they are based.

TABLE 8
Binding affinity of Karunyam proteins at 37° C.

| | Anti-virus | | |
|---|---|---|---|
| Karunyam | moiety | Spike protein | K_{D} (nM) |
| Karunyam 13 | scFv 13 | Delta RBD-spike protein | 14.64 ± 0.25 |
| Karunyam 14 | scFv 14 | Delta RBD-spike protein | 14.07 ± 0.29 |
| Karunyam 15 | scFv 15 | Delta RBD-spike protein | 16.41 ± 1.39 |
| Karunyam 18 | scFv 18 | Omicron RBD-spike protein | 12.81 ± 0.14 |
| Karunyam 19 | scFv 19 | Omicron RBD-spike protein | 13.10 ± 0.25 |
| Karunyam 20 | scFv 20 | Omicron RBD-spike protein | 12.98 ± 1.18 |

TABLE 9
Binding affinity of known antibodies of the prior art at 37° C.

| | | K_{D} against RBD-spike | |
|---|---|---|---|
| | Parent | protein or EC50 for virus | |
| Anti-virus moiety | antibody | isolates reported | Reference |
| scFv 13 | SARS 2-38 | N/A | 11 |
| scFv 14 | CT-P59 | 0.54 nM (to Delta RBD-spike | 12, 13 |
| | | protein); 0.055 nM (to SARS- | |
| | | CoV2-RBD) | |
| scFv 15 | LY-CoV1404 | 0.12 nM (to Delta RBD-spike | 14 |
| | | protein with D614G) | |
| scFv 18 | S2X259 | 0.2 nM (to SARS-CoV2- | 15 |
| | | RBD) | |
| scFv 19 | S2H97 | 414 ng/ml IC50 (to Omicron | 16 |
| | | pseudovirus) | |
| scFv 20 | S2K146 | 0.066 nM (to SARS-CoV2- | 17 |
| | | RBD) | |
| ACE2 receptor | N/A | 4.7 nM to SARS-CoV2-RBD; | 5 |
| | | 31 nM to SARS-CoV-RBD | |

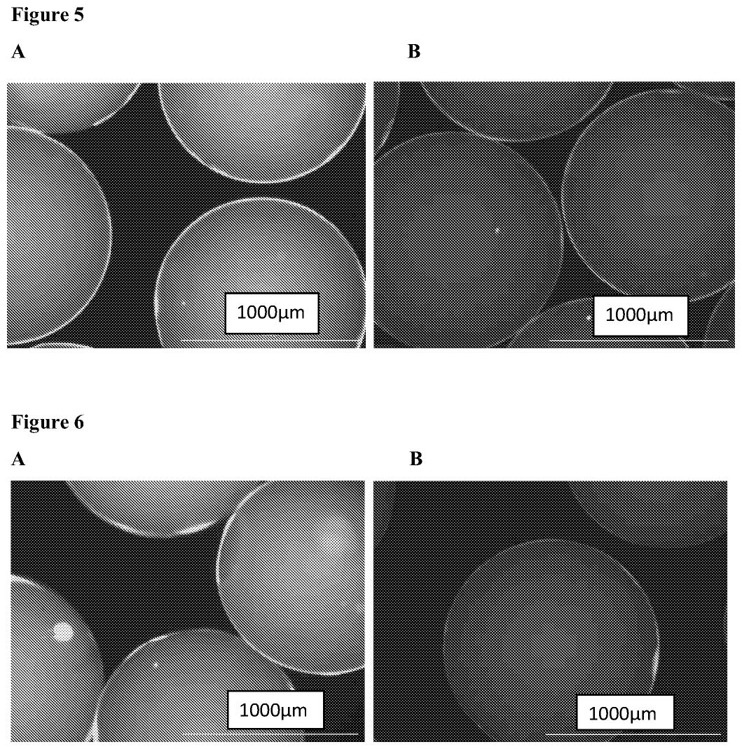

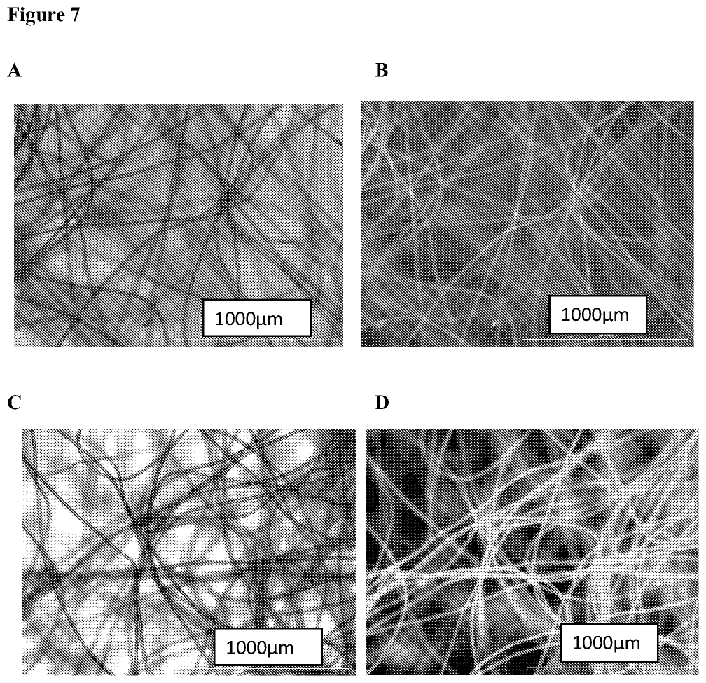

To test whether the Karunyam proteins targeting Delta and Omicron variants of SARS-COV-2 when bound to a cellulose matrix can bind to virus pseudoparticles, cellulose bead binding assays were performed. Karunyam proteins immobilised on cellulose beads were treated with serial dilutions of Delta, Delta-plus or Omicron RBD-spike protein conjugated with FITC nanoparticles. The FITC nanoparticles have a diameter of 100 nm so are representative of coronavirus particles, which are 60-140 nm in diameter. As shown in FIGS. 5 and 6, fluorescence of the cellulose beads was detected after 5 minutes, demonstrating that the anti-Delta Karunyam proteins bound to the cellulose beads can bind both the Delta RBD-spike protein conjugated with FITC nanoparticles and the Delta-plus RBD-spike protein conjugated with FITC nanoparticles. When left in solution for one week, the anti-Delta Karunyam proteins bound to the cellulose beads continued to bind the Delta or Delta-plus RBD-spike protein.

To estimate the amount of Delta, Delta-plus or Omicron RBD-spike protein bound to the Karunyam proteins immobilised on the cellulose beads, IC strips were used to firstly estimate the amount of total RBD-spike protein in the serial dilutions, and then to estimate the amount of unbound RBD-spike protein after addition to the beads. The difference between total and unbound RBD-spike protein provided the approximate amounts of RBD-spike protein bound to immobilised Karunyam proteins, as shown in Table 10, Table 11 and Table 12.

TABLE 10
Estimated amount of Delta RBD-spike protein bound to Karunyam
proteins immobilised on cellulose beads after 15 minutes.

| | Dilution of Delta RBD-spike protein | | | | | | |
|---|---|---|---|---|---|---|---|
| | 10 nM | 5 nM | 2.5 nM | 1.25nM | 0.63 nM | 0.31 nM | 0.15 nM |
| TOTAL | + | + | + | + | + (0.63) | − | − |
| Karunyam 13-unbound | + | + (0.63) | − | − | − | − | − |
| Karunyam 14-unbound | + | + (0.63) | − | − | − | − | − |
| Karunyam 15-unbound | + | + (0.63) | − | − | − | − | − |

The + sign indicates the presence of a positive signal on the IC strip.
The − sign indicates absence of a signal on the IC strip.
Estimation of fraction bound by 200 ng scFv (average): (5 − 0.63)/5 = 0.874.

TABLE 11
Estimated amount of Delta-plus RBD-spike protein bound to Karunyam
proteins immobilised on cellulose beads after 15 minutes.

| | Dilution of Delta RBD-spike protein | | | | | | |
|---|---|---|---|---|---|---|---|
| | 10 nM | 5 nM | 2.5 nM | 1.25 nM | 0.63 nM | 0.31 nM | 0.15 nM |
| TOTAL | + | + | + | + | + (0.63) | − | − |
| Karunyam 13-unbound | + | + (0.63) | − | − | − | − | − |
| Karunyam 14-unbound | + | + (0.63) | − | − | − | − | − |
| Karunyam 15-unbound | + | + (0.63) | − | − | − | − | − |

The + sign indicates the presence of a positive signal on the IC strip.
The − sign indicates absence of a signal on the IC strip.
Estimation of fraction bound by 200 ng scFv (average): (5 − 0.63)/5 = 0.874.

TABLE 12
Estimated amount of Omicron RBD-spike protein bound to Karunyam
proteins immobilised on cellulose beads after 15 minutes.

| | Dilution of Omicron RBD-spike protein | | | | | | |
|---|---|---|---|---|---|---|---|
| | 10 nM | 5 nM | 2.5 nM | 1.25 nM | 0.63 nM | 0.31 nM | 0.15 nM |
| TOTAL | + | + | + | + | + (0.63) | − | − |
| Karunyam 18-unbound | + | + (0.63) | − | − | − | − | − |
| Karunyam 19-unbound | + | + (0.63) | − | − | − | − | − |
| Karunyam 20-unbound | + | + (0.63) | − | − | − | − | − |

The + sign indicates the presence of a positive signal on the IC strip.
The − sign indicates absence of a signal on the IC strip.
Estimation of fraction bound by 200 ng scFv (average): (5 − 0.63)/5 = 0.874.

### Example 6—Further Matrix Affinity Ligands

This experiment aimed to express and purify Karunyam proteins wherein each Karunyam protein contains an scFv coupled to a chitin-binding domain or a polyester affinity tag. The binding properties of each of the Karunyam proteins were also investigated. This experiment also investigated the extent to which the Karunyam proteins, when immobilised on Chitosan or polyester fibre, can retain virus pseudoparticles.

### Materials and Methods

### DNA Constructs

Karunyam 9 and Karunyam 13 were expressed according the methods above, except each scFv was coupled to a different matrix-binding domain, specifically a chitin-binding domain (SEQ ID NO: 124) or a polyester affinity tag (Tachystatin A2) (SEQ ID NO: 125). Accordingly, four Karunyam proteins were prepared, two containing a scFv coupled to a chitin-binding domain, and two containing a scFv coupled to Tachystatin A2 (TA2). The resulting Karunyam proteins have the sequences set out in Table 13.

TABLE 13
Features of the Karunyam proteins.

| | | | Matrix binding |
|---|---|---|---|
| Karunyam protein | Anti-virus moiety | N-terminal tag | protein at C terminus |
| Karunyam 21 | scFv 9 | His-tag | Chitin-binding domain |
| (SEQ ID NO: 126) | (SEQ ID NO: 1) | (SEQ ID NO: 4) | (SEQ ID NO: 124) |
| Karunyam 22 | scFv 13 | and | |
| (SEQ ID NO: 127) | (SEQ ID NO: 46) | GST-tag | |
| Karunyam 23 | scFv 9 | (SEQ ID NO: 5) | TA2 (SEQ ID NO: 125) |
| (SEQ ID NO: 128) | (SEQ ID NO: 1) | | |
| Karunyam 24 | scFv 13 | | |
| (SEQ ID NO: 129) | (SEQ ID NO: 46) | | |

### Protein Expression and Affinity Purification

Karunyam proteins were expressed and affinity purified according to the methods described in Example 1.

### Enzyme-Linked Immunosorbent Assay (ELISA)

ELISA experiments were performed according to the method described in Example 1, except a biotinylated Delta RBD-spike protein from Sinobiological, amino acids Arg319-Phe541, was used in experiments using the Karunyam proteins that target the Delta variant of SARS-COV-2 (Karunyam proteins 22 and 24).

### Chitosan and Polyester Fibre Binding Assays

A square portion of Chitosan dressing, 1 cm×1 cm (ChitoSAM 100 dressing, SAM medical products, Wilsonville, OR, USA) or 100 mg of 100% polyester fibre (Fairfield Processing Corp, Danbury, CT, USA) were coated overnight with purified Karunyam protein at 4° C., using 200 ng of Karunyam protein in a total volume of 1 mL PBS in each well of a 12 well culture plate. One in two serial dilutions were prepared of RBD-spike protein conjugated to FITC nanoparticles (viral pseudoparticles). 60 μL of each dilution was estimated for RBD-spike protein using immunochromatography (IC) strips. The reactivity was estimated after 15 minutes. The Chitosan dressing or polyester fibre was washed twice with TBST and then treated for one hour at 37° C. with serial dilutions of RBD-spike protein conjugated with FITC nanoparticles. The unbound RBD-spike protein content was estimated using IC strips. For RBD-spike protein content, RBD spike protein dilutions in 100 μL TBST were prepared ranging from 20 nM to 0.15 nM. 60 μL of each dilution was spotted on the IC strip and allowed to develop over 15 minutes. The presence of the positive band indicates the presence of RBD-spike protein. The threshold sensitivity of the IC strip was determined under these conditions as 0.63 nM.

The Chitosan dressing or polyester fibre with immobilised Karunyam protein were added to the serial dilutions of RBD-spike protein. The RBD-spike protein dilutions were then re-tested using the IC strips to estimate the amount of unbound RBD-spike protein after treatment with the Chitosan dressing or polyester fibre. Table 16 shows the serial dilutions of RBD-spike protein and the qualitative IC strip results before and after treatment with immobilised Karunyam proteins. The difference in IC testing between pre- and post-Karunyam treatment RBD-spike protein provided the approximate amounts of RBD-spike protein bound to immobilised Karunyam proteins.

Uncoated Chitosan dressing and uncoated polyester fibre were used as negative controls.

### Results

To quantify the binding affinity of the Karunyam proteins containing the chitin-binding domain or TA2 to the RBD-spike protein and the RBD-spike protein of the Delta variant of SARS-COV-2, ELISA experiments were performed according to the method described above. K_{D} value for each Karunyam protein was estimated from dose response curves, and are shown in Table 14 below. For comparison, Table 15 shows the K_{D} values for the known antibodies from which the scFvs were derived. As shown in Table 14, each of Karunyam 21 and Karunyam 23 binds to the original RBD-spike protein with high affinity and each of Karunyam 22 and Karunyam 24 binds to the Delta RBD-spike protein with high affinity.

TABLE 14
Binding affinity of Karunyam proteins at 37° C.

| | Anti-virus | | |
|---|---|---|---|
| Karunyam | moiety | Spike protein | K_{D} (nM) |
| Karunyam 21 | scFv 9 | Original RBD-spike protein | 12.77 ± 0.24 |
| Karunyam 22 | scFv 13 | Delta RBD-spike protein | 13.29 ± 1.13 |
| Karunyam 23 | scFv 9 | Original RBD-spike protein | 12.26 ± 0.22 |
| Karunyam 24 | scFv 13 | Delta RBD-spike protein | 15.59 ± 0.65 |

TABLE 15
Binding affinity of known antibodies of the prior art at 37° C.

| | | K_{D} against Spike protein | |
|---|---|---|---|
| Anti-virus moiety | Parent antibody | (nM) | Reference |
| scFv 9 | P2B 2F6 | 5.14 nM | 6 |
| scFv 13 | SARS 2-38 | N/A | 6 |

TABLE 16
Estimated amount of RBD-spike protein bound to Karunyam
proteins immobilised on cellulose beads after 15 minutes.

| | Dilution of RBD-spike protein | | | | |
|---|---|---|---|---|---|
| | 20 | 10 | 5 | 2.5 | Estimation of fraction bound |
| | nM | nM | nM | nM | by 200 ng scFv (average) |
| TOTAL | + | + | + | + | |
| Karunyam 21- | + | + | + | − | LOW |
| unbound | | | | | |
| Karunyam 22- | + | + | + | − | LOW |
| unbound | | | | | |
| Karunyam 23- | − | − | − | − | 100% |
| unbound | | | | | |
| Karunyam 24- | − | − | − | − | 100% |
| unbound | | | | | |

The + sign indicates the presence of a positive signal on the IC strip.
The − sign indicates absence of a signal on the IC strip.

### Example 7—HACE2 as Anti-Virus Moiety

This experiment aims to express and purify Karunyam proteins that utilise decoy receptors as the virus binding domain, such as the human ACE2 receptor and mutants thereof. The binding properties of each of the Karunyam proteins are also investigated. This experiment also investigates the extent to which the Karunyam proteins, when immobilised on cellulose beads, can retain virus pseudoparticles.

### Materials and Methods

### DNA Constructs

Six Karunyam proteins, each containing a 131 amino acid fragment of the ACE2 receptor coupled to a matrix-binding domain (cellulose-binding motif 3, CBM3), were prepared. ACE2 receptor fragment sequences were based on known sequences of the human ACE2 receptor (hACE2) and mutants thereof, as disclosed in Reference 18. The resulting Karunyam proteins have the sequences set out in Table 17.

TABLE 17
Features of the Karunyam proteins.

| | | | Matrix binding |
|---|---|---|---|
| Karunyam protein | Anti-virus moiety | N-terminal tag | protein at C terminus |
| Karunyam 25 | Wild-type hACE2 | His-tag | CBM3 (SEQ ID NO: 6) |
| (SEQ ID NO: 135) | (R148) (SEQ ID | (SEQ ID NO: 4) | |
| | NO: 130) | and | |
| Karunyam 26 | M1 (SEQ ID NO: | GST-tag | |
| (SEQ ID NO: 136) | 131) | (SEQ ID NO: 5) | |
| Karunyam 27 | M2.1 (SEQ ID NO: | | |
| (SEQ ID NO: 137) | 132) | | |
| Karunyam 28 | M2.2 (SEQ ID NO: | | |
| (SEQ ID NO: 138) | 133) | | |
| Karunyam 29 | M3 (SEQ ID NO: | | |
| (SEQ ID NO: 139) | 134) | | |
| Karunyam 30 | M4 (SEQ ID NO: | | |
| (SEQ ID NO: 140) | 135) | | |

hACE2 R148 corresponds to amino acid positions 18 to 148 of the mature human ACE2 receptor. The protein was obtained from the human ACE2 receptor cDNA containing plasmid (Genscript OHu20260D, Genbank accession number NM 021804.2, nucleotides 269-662).

hACE2 M1 was obtained by point mutation PCR using R148 as a template, introducing the mutations K31F/H34I/E35Q.

hACE M2.1 was obtained by point mutation PCR using R148 as a template, introducing the mutation T27Y.

hACE M2.2 was obtained by point mutation PCR using M2.1 as a template, introducing the mutation L79T.

hACE M3 was obtained by point mutation PCR using M2.1 as a template, introducing the mutation T92Q.

hACE M4 was obtained by point mutation PCR using M2.1 as a template, introducing the mutation H34A.

### Protein Expression and Affinity Purification

Karunyam proteins were expressed and affinity purified according to the methods described in Example 1.

### Enzyme-Linked Immunosorbent Assay (ELISA)

ELISA experiments were performed according to the method described in Example 1.

### Cellulose Bead Binding Assay

Cellulose bead binding assays were performed according to the method described in Example 2.

### Results

To estimate the amount of RBD-spike protein bound to the Karunyam proteins immobilised on the cellulose beads, IC strips were used to firstly estimate the amount of total RBD-spike protein in the serial dilutions, and then to estimate the amount of unbound RBD-spike protein after addition to the beads. The difference between total and unbound RBD-spike protein provided the approximate amounts of RBD-spike protein bound to immobilised Karunyam proteins, as shown in Table 18. Estimation of fraction bound by the anti-virus moiety is also provided. For example, the estimated fraction bound by 200 ng R148WT is calculated by 20−2.5/20=0.875. Each of Karunyam 25 to 30 retained a large proportion of the total virus pseudoparticles after centrifugation.

TABLE 18
Estimated amount of RBD-spike protein bound to Karunyam
proteins immobilised on cellulose beads after 15 minutes.

| | Dilution of RBD-spike protein | | | | |
|---|---|---|---|---|---|
| | | | | | Estimation of fraction |
| | 10 | 5 | 2.5 | 1.25 | bound by 200 ng scFv |
| | nM | nM | nM | nM | (average) |
| TOTAL | + | + | + | − | 0.875 (high) |
| | | | (2.5) | | |
| Karunyam 25- | + | + | − | − | 0.875 (high) |
| unbound | | (2.5) | | | |
| Karunyam 26- | + | + | − | − | 0.875 (high) |
| unbound | | (2.5) | | | |
| Karunyam 27- | + | + | − | − | 0.875 (high) |
| unbound | | (2.5) | | | |
| Karunyam 28- | + | + | − | − | 0.875 (high) |
| unbound | | (2.5) | | | |
| Karunyam 29- | + | + | − | − | 0.875 (high) |
| unbound | | (2.5) | | | |
| Karunyam 30- | + | + | − | − | 0.875 (high) |
| unbound | | (2.5) | | | |

The + sign indicates the presence of a positive signal on the IC strip.
The − sign indicates absence of a signal on the IC strip.

To quantify the binding affinity of the Karunyam proteins to the RBD-spike protein of SARS-COV-2, ELISA experiments were performed according to the method described above. K_{D} values for each Karunyam protein were estimated from dose response curves, and are shown in Table 19 below. For comparison, Table 20 shows the K_{D} values for the wild-type hACE2 receptor fragment and the mutant receptor fragments thereof. As shown in Table 19, each of Karunyam 25, 26, 27, 28, 29 and 30 binds to the RBD-spike protein with high affinity.

TABLE 19
Binding affinity of Karunyam proteins at 37° C.

| | hACE2 | Sequence alteration from | K_{D} against RBD-spike |
|---|---|---|---|
| Karunyam | fragment | hACE2 | protein (nM) |
| Karunyam | R148 | WT | 18.18 ± 0.39 |
| 25 | | | |
| Karunyam | M1 | K31F/H34I/E35Q | 17.01 ± 0.43 |
| 26 | | | |
| Karunyam | M2.1 | T27Y | 17.39 ± 0.80 |
| 27 | | | |
| Karunyam | M2.2 | T27Y/L79T | n.d. |
| 28 | | | |
| Karunyam | M3 | T27Y/T92Q | 16.79 ± 0.36 |
| 29 | | | |
| Karunyam | M4 | T27Y/H34A | n.d. |
| 30 | | | |

TABLE 20
Binding affinity of wild-type ACE2 fragment from Reference 4

| | | K_{D} against RBD-spike |
|---|---|---|
| hACE2 fragment | Mutation | protein (nM) |
| R148 (wild-type) | N/A (wild-type) | 140 |
| M1 | K31F/H34I/E35Q | N/A |
| M2.1 | T27Y | 2.3 |
| M2.2 | T27Y/L79T | N/A |
| M3 | T27Y/L92Q | N/A |
| M4 | T27Y/H34A | N/A |

### Example 8

This experiment investigated the extent to which Karunyam proteins when immobilised on materials, can retain virus pseudoparticles. In particular, Karunyam proteins that bind to cellulose beads, Chitosan fabric and polyester fibre were tested.

### Materials and Methods

### Virus Pseudoparticle Trap (Spin Column) Assay

Karunyam proteins bound to different matrices (cellulose beads, chitosan dressing, or polyester fibre) were subject to the virus pseudoparticle trap (spin column) assay. In detail, cellulose beads of 1 mm average diameter (100 mg), chitosan dressing (1 cm×1 cm), or 100% polyester fibre (100 mg) were coated overnight with purified Karunyam protein at 4° C., using 200 ng of Karunyam protein in a total volume of 0.5 mL PBS in a spin-column. RBD-spike protein conjugated to FITC nanoparticles (virus pseudoparticles) at a concentration of 10 nM were incubated in each spin column in a volume of 200 μL for 30 minutes at 37° C. The spin columns were centrifuged at 14,000 g for 5 minutes. The unbound RBD-spike protein fraction was determined using a IC assay, using serial dilutions. The difference between total and unbound RBD-spike protein as provided by IC analysis provides a value of the RBD-spike protein bound to the matrix.

To determine the saturation of the polyester-bound Karunyam 23 trap, increasing amounts of virus pseudoparticles were bound to the columns as above (corresponding to 60-300 ng RBD-spike protein). Even at the highest value of 300 ng, there was no visible amount of viral pseudoparticle detected by IC analysis in the eluate, indicating that saturation had not yet been reached.

### Results

Table 21 shows the features of the Karunyam proteins used in the virus pseudoparticle trap assay, in particular the sequence of the anti-virus moiety, the matrix binding protein at the C-terminus and the spike protein that the anti-virus moiety recognises.

The results of the virus pseudoparticle trap assay are also shown in Table 22. Karunyam 9, Karunyam 23, Karunyam 13 and Karunyam 24 all retained a large proportion of the total virus pseudoparticles after centrifugation.

These results show that Karunyam 23 and Karunyam 24 immobilised on polyester fibre are able to retain very high amounts of virus pseudoparticles after centrifugation. Accordingly, these proteins can act as “traps” for virus particles and/or fragments thereof.

TABLE 21
Features of the Karunyam proteins and the estimated amount
of RBD-spike protein bound to Karunyam proteins.

| | | Matrix binding | | Binding in |
|---|---|---|---|---|
| Karunyam | Anti-virus | protein at C | | spin column |
| protein | moiety | terminus | Spike protein | assays |
| Karunyam 9 | scFv 9 (SEQ | CBM3 (SEQ ID | Original RBD- | 87% |
| (SEQ ID NO: | ID NO: 1) | NO: 6) | spike protein | |
| 7) | | | | |
| Karunyam 21 | scFv 9 (SEQ | Chitin-binding | Original RBD- | <10% |
| (SEQ ID NO: | ID NO: 1) | domain (SEQ ID | spike protein | |
| 125) | | NO: 124) | | |
| Karunyam 23 | scFv 9 (SEQ | TA2 (SEQ ID | Original RBD- | 100% |
| (SEQ ID NO: | ID NO: 1) | NO: 125) | spike protein | |
| 127) | | | | |
| Karunyam 13 | scFv 13 (SEQ | CBM3 (SEQ ID | Delta RBD- | 87% |
| (SEQ ID NO: | ID NO: 46) | NO: 6) | spike protein | |
| 49) | | | and Delta-plus | |
| | | | RBD-spike | |
| | | | protein | |
| Karunyam 22 | scFv 13 (SEQ | Chitin-binding | Delta RBD- | <10% |
| (SEQ ID NO: | ID NO: 46) | domain (SEQ ID | spike protein | |
| 127) | | NO: 124) | | |
| Karunyam 24 | scFv 13 (SEQ | TA2 (SEQ ID | Delta RBD- | 100% |
| (SEQ ID NO: | ID NO: 46) | NO: 125) | spike protein | |
| 129) | | | | |

TABLE 22
Estimated amount of RBD-spike protein bound to Karunyam 23 immobilised on matrix after 15 minutes.

| | | | | 20 nM × | 20 nM × | 20 nM × | 20 nM × | 20 nM × |
|---|---|---|---|---|---|---|---|---|
| | scFv | Tag | Spike protein | 100 μL | 200 μL | 300 μL | 400 μL | 500 μL |
| RBD-spike loaded (ng) | | | | 60 | 120 | 180 | 240 | 300 |
| Total | | | | + | + | + | + | + |
| Unbound Karunyam 23 | scFv 9 | TA2 | Original RBD-spike protein | − | − | − | − | − |

### Example 9—Stability of Karunyam Proteins

This experiment investigated the stability and extended storage life of the Karunyam proteins of the invention.

### Materials and Methods

### Virus Pseudoparticle Binding Assay Under Different Storage Conditions

Cellulose beads of 1 mm average diameter were coated for one week with purified Karunyam protein at 4° C. and at 37° C., using 200 ng of Karunyam protein in a total volume of 1 mL PBS in each well of a 12 well culture plate. Cellulose bead binding assays were carried out as described in Example 2, using IC as binding indicators.

### Results

To test whether the Karunyam 9 when bound to a cellulose matrix for extended periods of time can still bind to virus pseudoparticles, cellulose bead binding assays were performed according to Example 2, except beads were first coated with Karunyam for one week at 4° C. and at 37° C. Karunyam proteins immobilised on cellulose beads for one week were then treated with serial dilutions of RBD-spike protein conjugated with FITC nanoparticles.

To estimate the amount of RBD-spike protein bound to the Karunyam proteins immobilised on the cellulose beads, IC strips were used to firstly estimate the amount of total RBD-spike protein in the serial dilutions, and then to estimate the amount of unbound RBD-spike protein after addition to the beads. The difference between total and unbound RBD-spike protein provided the approximate amounts of RBD-spike protein bound to immobilised Karunyam proteins, as shown in Table 23.

TABLE 23
Estimated amount of RBD-spike protein bound to Karunyam 9 immobilised
on cellulose beads under different storage conditions.

| | | | Dilution of original RBD-spike protein | | | | | |
|---|---|---|---|---|---|---|---|---|
| Storage | | | 10 nM | 5 nM | 2.5 nM | 1.25 nM | 0.63 nM | 0.31 nM |
| | | TOTAL | + | + | + | + | + (0.63) | − |
| 15 | minutes | Karunyam 9-unbound | + | + (0.63) | − | − | − | − |
| 4° C. | one week | Karunyam 9-unbound | + | + (0.63) | − | − | − | − |
| 37° C. | one week | Karunyam 9-unbound | + | + | + | + | + | − |

The + sign indicates the presence of a positive signal on the IC strip.
The − sign indicates absence of a signal on the IC strip.

### Example 10—Removal of Virus Trapped by Karunyam Proteins

This experiment investigated the removal of virus pseudoparticles trapped by immobilised Karunyam proteins of the invention. In detail, 100 mg polyester fibre was coated overnight with Karunyam 23 at 4° C., using 200 ng of Karunyam 23 in a total volume of 0.5 mL PBS in a spin-column. RBD-spike protein conjugated to FITC nanoparticles (virus pseudoparticles) at a concentration of 10 nM were incubated in each spin column in a volume of 200 μL for 30 minutes at 37° C. The spin columns were centrifuged at 14,000 g for 1 minute. 0.5 mL PBS was added to the spin columns and the spin columns were centrifuged for 1 minute to wash the polyester fibre. The wash step was repeated once. The polyester fibres were transferred to 1.5 mL microcentrifuge tubes and incubated for 1 hour at 37° C., in different detergent solutions (2% Sodium lauroyl sarcosinate, 2% SDS, 2% Triton X-100, 2% Tween-20, respectively). Polyester fibres were transferred to 12-well tissue culture plates and photographed for remnant fluorescence.

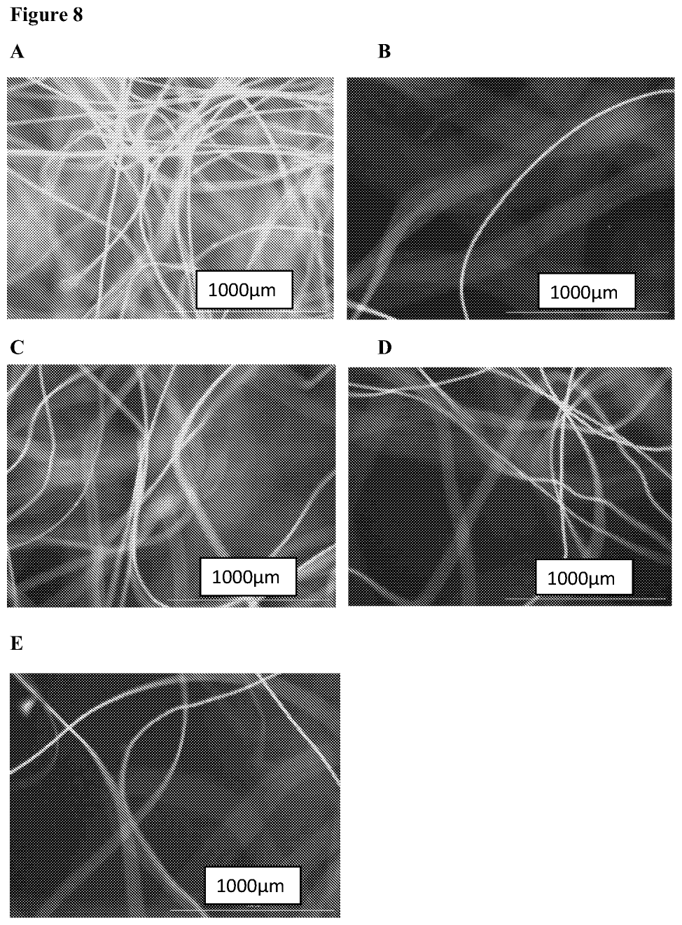

As shown in FIG. 8, fluorescence signal in B-E is decreased compared to A but signal still observable after incubation with each detergent solution at 37° C., indicating that polyester fibre retains some virus pseudoparticles after washing with detergent solutions at 37° C.

Therefore different conditions were investigated. After the repeated wash step outlined above, the polyester fibres were transferred to 1.5 mL microcentrifuge tubes and incubated for 1 hour at 100° C., in different detergent solutions (2% Sodium lauroyl sarcosinate, 2% Triton X-100, 2% Tween-20, distilled water and 10% HOAc, 50% MeOH).

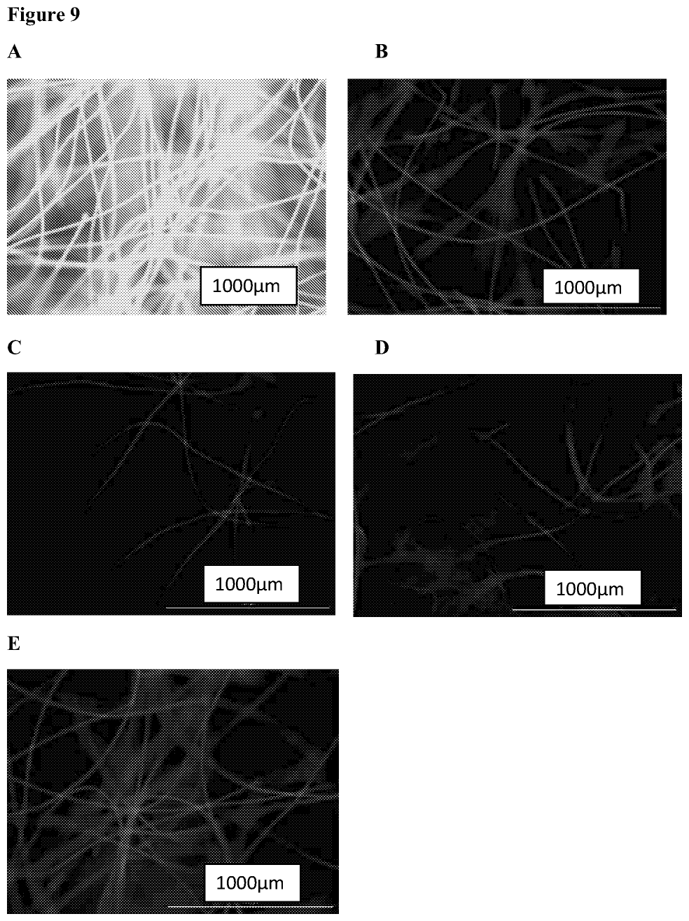

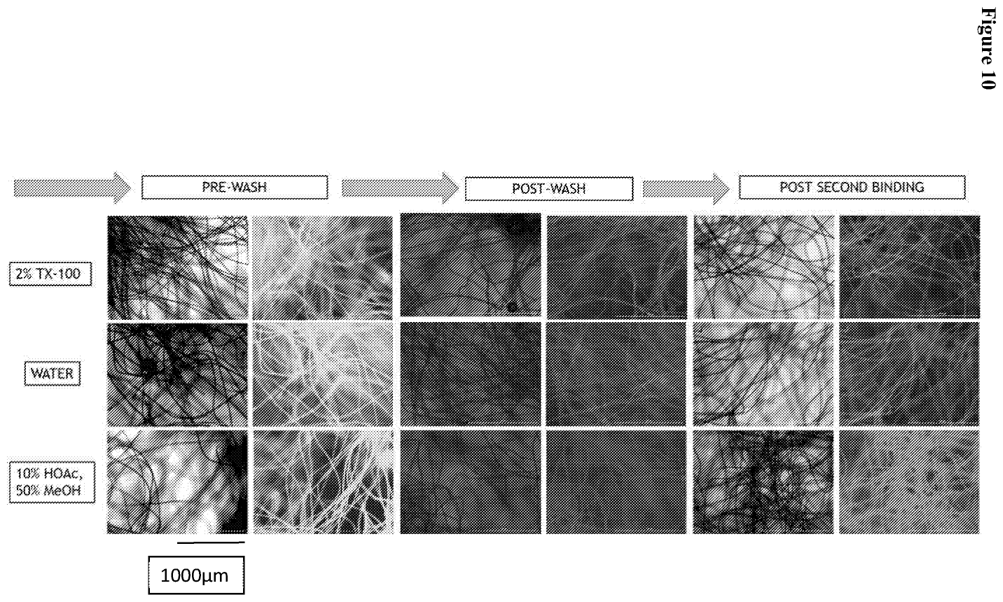

As shown in FIG. 9, fluorescence signal in B-E decreases significantly compared to A, i.e. after incubation with water or detergent solutions at 100° C., indicating that virus pseudoparticles are substantially removed from polyester fibre after washing with water or detergent solutions at 100° C.

### Example 11—Re-Usability of Karunyam Proteins and Matrices

This experiment investigated the re-usability of the Karunyam proteins of the invention. In detail, 100 mg polyester fibre was coated overnight with Karunyam 23 at 4° C., using 200 ng of Karunyam 23 in a total volume of 0.5 mL PBS in a spin-column. RBD-spike protein conjugated to FITC nanoparticles (virus pseudoparticles) at a concentration of 10 nM were incubated in each spin column in a volume of 200 μL for 30 minutes at 37° C. The spin columns were centrifuged at 14,000 g for 1 minute. 0.5 mL PBS was added to the spin columns and the spin columns were centrifuged for 1 minute to wash the polyester fibre. The wash step was repeated once. The polyester fibres were transferred to 12-well tissue culture plates in 1 mL PBS for photography (labelled PRE-WASH). The polyester fibres were transferred to 1.5 mL microcentrifuge tubes and incubated for 1 hour at 100° C., in different detergent solutions (2% Triton X-100, distilled water, 10% HOAc, 50% MeOH). Following incubation, the polyester fibres were washed twice with 0.5 mL PBS and photographed (labelled POST-WASH). The polyester fibres were re-incubated with Karunyam 23 using 200 ng of Karunyam 23 in a total volume of 0.5 mL PBS in a spin-column. RBD-spike protein conjugated to Texas red nanoparticles (virus pseudoparticles) at a concentration of 10 nM were incubated in each spin column in a volume of 200 μL for 30 minutes at 37° C. The spin columns were centrifuged at 14,000 g for 1 minute. 0.5 mL PBS was added to the spin columns and the spin columns were centrifuged for 1 minute to wash the polyester fibre. The wash step was repeated once. The polyester fibres were transferred to 12-well tissue culture plates in 1 mL PBS for photography (labelled POST SECOND BINDING).

As shown in FIG. 10, fluorescence signal at the PRE-WASH stage is stronger than fluorescence signal at the POST-WASH stage, i.e. after incubation with each detergent solution at 100° C. As concluded in Example 11, virus pseudoparticles are substantially removed from polyester fibre after washing with detergent solutions at 100° C. At the POST SECOND BINDING stage, red fluorescence signal increases (see last column in FIG. 10), indicating that the polyester fibres are bound by the RBD-spike protein conjugated to Texas red nanoparticles (virus pseudoparticles).

Therefore, matrices that have been previously bound by Karunyam proteins can be reused to bind new Karunyam proteins.

### REFERENCES

1 Holliger and Hudson, (2005) Nature Biotech. 23 (9): 1126-1136.
2 Adair and Lawson, (2005) Drug Design Reviews-Online 2 (3), 209-217.
3 Verma et al., (1998) Journal of Immunological Methods, 216, 165-181.
4 Chan et al., (2020) Science, 369 (6508): 1261-1265.
5 Lan et al., (2020) Nature, 581:215-220.
6 Ju et al., (2020) Nature, 584:115-119.
7 Chong et al., (1997) Gene, 192 (2): 271-281.
8 Rubsam et al., (2017) Biotech Bioeng, 115 (2): 321-330.
9 Zhou et al., (2021) J Aerosol Sci, 152:105693.
10 Malik et al., (2021) Int. J. Infect. Dis., 110:105-110.
11 Van Blargan et al., (2021) Immunity, 54 (10): 2399-2416.
12 Kim et al. (2021) Nat Commun., 12, 288.
13 Ryu et al. (2021) Biochem. Biophys. Res. Commun. 578, 91-96.
14 Westendorf (2022) Cell Rep. 39, 7.
15 Tortorici et al., (2021) Nature, 597, 103-108.
16 Starr et al., (2021) Nature, 597, 97-102.
17 Park et al., (2022) Science, 375, 449-454.
18 Chan et al., (2020) Science, 369 (6508): 1261-1265.

### SEQUENCE LISTING

SEQ ID NO: 1-scFv 9
DPIVMIDNDKPITGGGGSGSQVQLQESGPGLVKPSETLSLTCTVSGYSISSGYYWG
WIRQPPGKGLEWIGSIYHSGSTYYNPSLKTRVTISVDTSKNQFSLKLSSVTAADTAV
YYCARAVVGIVVVPAAGRRAFDIWGQGTMVTVSSGGGGSGGGGSGGGGSGGGG
SGGGGSQSALTQPPSASGSPGQSVTISCTGTSSDVGGYNYVSWYQQHPGKAPKLM
IYEVSKRPSGVPDRFSGSKSGNTASLTVSGLQAEDEADYYCSSYAGSNNLVFGGGT
KV
SEQ ID NO: 2-scFv 10
AHIVMVDAYKPTKGGGGSGSEVOLVESGGGLVQPGGSLRLSCAASGITVSSNYM
NWVRQAPGKGLEWVSLIYSGGSTYYADSVKGRFTISRDNSKNTLYLQMNSLRAE
DTAVYHCARDLVVYGMDVWGQGTTVTVSSGGGGSGGGGSGGGGSGGGGSGGG
GSEIVLTQSPGTLSLSPGERATLSCRASQSVSSSYLAWYQQKPGQAPRLLIYGASSR
ATGIPDRFSGSGSGTDFTLTISRLEPEDFAVYYCQQYGSSPTFGQGTKLEIK
SEQ ID NO: 3-scFv 11
KLGDIEFIKVNKGGGGSGSQVQLVESGGGLVKPGGSLRLSCAASGFTFSDYYMSW
IRQAPGKGLEWVSYISSSGSTIYYADSVKGRFTISRDNAKNSLYLQMNSLRAEDTA
VYYCARDFSHQQLVPSWGQGTLVTVSSGGGGSGGGGSGGGGSGGGGSGGGGSDI
QLTQSPSFLSASVGDRVTITCRASQGISSYLAWYQQKPGKAPKLLIYAASTLQSGV
PSRFSGSGSGTEFTLTISSLQPEDFATYYCQQLNSYPLTFGGGTKVEIK
SEQ ID NO: 4-Histidine affinity tag
HHHHHH
SEQ ID NO: 5-Glutathione-S-transferase (GST) solubility tag.
SPILGYWKIKGLVQPTRLLLEYLEEKYEEHLYERDEGDKWRNKKFELGLEFPNLPY
YIDGDVKLTQSMAIIRYIADKHNMLGGCPKERAEISMLEGAVLDIRYGVSRIAYSK
DFETLKVDFLSKLPEMLKMFEDRLCHKTYLNGDHVTHPDFMLYDALDVVLYMDP
MCLDAFPKLVCFKKRIEAIPQIDKYLKSSKYIAWPLQGWQATFGGGDHPPKGIEEN
LYFQS
SEQ ID NO: 6-Cellulose-binding domain 3(CBM3)
VSGNLKVEFYNSNPSDTTNSINPQFKVTNTGSSAIDLSKLTLRYYYTVDGQKDQTF
WCDHAAIIGSNGSYNGITSNVKGTFVKMSSSTNNADTYLEISFTGGTLEPGAHVQI
QGRFAKNDWSNYTQSNDYSFKSASQFVEWDQVTAYLNGVLVWGKESR
SEQ ID NO: 7-Karunyam 9
MNHKVHHHHHHIEGRHMSPILGYWKIKGLVQPTRLLLEYLEEKYEEHLYERDEG
DKWRNKKFELGLEFPNLPYYIDGDVKLTQSMAIIRYIADKHNMLGGCPKERAEIS
MLEGAVLDIRYGVSRIAYSKDFETLKVDFLSKLPEMLKMFEDRLCHKTYLNGDHV
THPDFMLYDALDVVLYMDPMCLDAFPKLVCFKKRIEAIPQIDKYLKSSKYIAWPL
QGWQATFGGGDHPPKGIEENLYFQSHMELGTLEGEFDPIVMIDNDKPITGGGGSGS
QVQLQESGPGLVKPSETLSLTCTVSGYSISSGYYWGWIRQPPGKGLEWIGSIYHSG
STYYNPSLKTRVTISVDTSKNQFSLKLSSVTAADTAVYYCARAVVGIVVVPAAGR
RAFDIWGQGTMVTVSSGGGGSGGGGSGGGGSGGGGSGGGGSQSALTQPPSASGS
PGQSVTISCTGTSSDVGGYNYVSWYQQHPGKAPKLMIYEVSKRPSGVPDRFSGSK
SGNTASLTVSGLQAEDEADYYCSSYAGSNNLVFGGGTKVDSVSGNLKVEFYNSNP
SDTTNSINPQFKVTNTGSSAIDLSKLTLRYYYTVDGQKDQTFWCDHAAIIGSNGSY
NGITSNVKGTFVKMSSSTNNADTYLEISFTGGTLEPGAHVQIQGRFAKNDWSNYT
QSNDYSFKSASQFVEWDQVTAYLNGVLVWGKESR
SEQ ID NO: 8-Karunyam 10
MNHKVHHHHHHIEGRHMSPILGYWKIKGLVQPTRLLLEYLEEKYEEHLYERDEG
DKWRNKKFELGLEFPNLPYYIDGDVKLTQSMAIIRYIADKHNMLGGCPKERAEIS
MLEGAVLDIRYGVSRIAYSKDFETLKVDFLSKLPEMLKMFEDRLCHKTYLNGDHV
THPDFMLYDALDVVLYMDPMCLDAFPKLVCFKKRIEAIPQIDKYLKSSKYIAWPL
QGWQATFGGGDHPPKGIEENLYFQSHMELGTLEGEFAHIVMVDAYKPTKGGGGS
GSEVOLVESGGGLVQPGGSLRLSCAASGITVSSNYMNWVRQAPGKGLEWVSLIYS
GGSTYYADSVKGRFTISRDNSKNTLYLQMNSLRAEDTAVYHCARDLVVYGMDV
WGQGTTVTVSSGGGGSGGGGSGGGGSGGGGSGGGGSEIVLTQSPGTLSLSPGERA
TLSCRASQSVSSSYLAWYQQKPGQAPRLLIYGASSRATGIPDRFSGSGSGTDFTLTI
SRLEPEDFAVYYCQQYGSSPTFGQGTKLEIKDSVSGNLKVEFYNSNPSDTTNSINPQ
FKVTNTGSSAIDLSKLTLRYYYTVDGQKDQTFWCDHAAIIGSNGSYNGITSNVKGT
FVKMSSSTNNADTYLEISFTGGTLEPGAHVQIQGRFAKNDWSNYTQSNDYSFKSA
SQFVEWDQVTAYLNGVLVWGKESR
SEQ ID NO: 9-Karunyam 11
MNHKVHHHHHHIEGRHMSPILGYWKIKGLVQPTRLLLEYLEEKYEEHLYERDEG
DKWRNKKFELGLEFPNLPYYIDGDVKLTQSMAIIRYIADKHNMLGGCPKERAEIS
MLEGAVLDIRYGVSRIAYSKDFETLKVDFLSKLPEMLKMFEDRLCHKTYLNGDHV
THPDFMLYDALDVVLYMDPMCLDAFPKLVCFKKRIEAIPQIDKYLKSSKYIAWPL
QGWQATFGGGDHPPKGIEENLYFQSHMELGTLEGEFKLGDIEFIKVNKGGGGSGS
QVQLVESGGGLVKPGGSLRLSCAASGFTFSDYYMSWIRQAPGKGLEWVSYISSSG
STIYYADSVKGRFTISRDNAKNSLYLQMNSLRAEDTAVYYCARDFSHQQLVPSWG
QGTLVTVSSGGGGSGGGGSGGGGSGGGGSGGGGSDIQLTQSPSFLSASVGDRVTIT
CRASQGISSYLAWYQQKPGKAPKLLIYAASTLQSGVPSRFSGSGSGTEFTLTISSLQ
PEDFATYYCQQLNSYPLTFGGGTKVEIKDSVSGNLKVEFYNSNPSDTTNSINPQFK
VTNTGSSAIDLSKLTLRYYYTVDGQKDQTFWCDHAAIIGSNGSYNGITSNVKGTFV
KMSSSTNNADTYLEISFTGGTLEPGAHVQIQGRFAKNDWSNYTQSNDYSFKSASQ
FVEWDQVTAYLNGVLVWGKESR
SEQ ID NO: 10-FR1 of the heavy chain variable domain of scFv 9
QVQLQESGPGLVKPSETLSLTCTVS
SEQ ID NO: 11-CDR1 of the heavy chain variable domain of scFv 9
GYSISSGYY
SEQ ID NO: 12-FR2 of the heavy chain variable domain of scFv 9
WGWIRQPPGKGLEWIGS
SEQ ID NO: 13-CDR2 of the heavy chain variable domain of scFv 9
IYHSGST
SEQ ID NO: 14-FR3 of the heavy chain variable domain of scFv 9
YYNPSLKTRVTISVDTSKNQFSLKLSSVTAADTAVYYC
SEQ ID NO: 15-CDR3 of the heavy chain variable domain of scFv 9
ARAVVGIVVVPAAGRRAFDIW
SEQ ID NO: 16-FR1 of the light chain variable domain of scFv 9
QSALTQPPSASGSPGQSVTISCTGT
SEQ ID NO: 17-CDR1 of the light chain variable domain of scFv 9
SSDVGGYNY
SEQ ID NO: 18-FR2 of the light chain variable domain of scFv 9
VSWYQQHPGKAPKLMIY
SEQ ID NO: 19-CDR2 of the light chain variable domain of scFv 9
EVS
SEQ ID NO: 20-FR3 of the light chain variable domain of scFv 9
KRPSGVPDRFSGSKSGNTASLTVSGLQAEDEADYYC
SEQ ID NO: 21-CDR3 of the light chain variable domain of scFv 9
SSYAGSNNLVFGGGTKVD
SEQ ID NO: 22-FR1 of the heavy chain variable domain of scFv 10
EVOLVESGGGLVQPGGSLRLSCAAS
SEQ ID NO: 23-CDR1 of the heavy chain variable domain of scFv 10
GITVSSNY
SEQ ID NO: 24-FR2 of the heavy chain variable domain of scFv 10
MNWVRQAPGKGLEWVSL
SEQ ID NO: 25-CDR2 of the heavy chain variable domain of scFv 10
IYSGGSTY
SEQ ID NO: 26-FR3 of the heavy chain variable domain of scFv 10
YADSVKGRFTISRDNSKNTLYLQMNSLRAEDTAVYHC
SEQ ID NO: 27-CDR3 of the heavy chain variable domain of scFv 10
ARDLVVYGMDVWGQGTTVTV
SEQ ID NO: 28-FR1 of the light chain variable domain of scFv 10
EIVLTQSPGTLSLSPGERATLSC
SEQ ID NO: 29-CDR1 of the light chain variable domain of scFv 10
RASQSVSSSY
SEQ ID NO: 30-FR2 of the light chain variable domain of scFv 10
LAWYQQKPGQAPRLLIY
SEQ ID NO: 31-CDR2 of the light chain variable domain of scFv 10
GAS
SEQ ID NO: 32-FR3 of the light chain variable domain of scFv 10
SRATGIPDRFSGSGSGTDFTLTISRLEPEDFAVYYCQQYGSSPTFGQGTKLEIKVD
SEQ ID NO: 33-CDR3 of the light chain variable domain of scFv 10
QQYGSSPTFGQGTKLEIKVD
SEQ ID NO: 34-FR1 of the heavy chain variable domain of scFv 11
QVQLVESGGGLVKPGGSLRLSCAAS
SEQ ID NO: 35-CDR1 of the heavy chain variable domain of scFv 11
GFTFSDYY
SEQ ID NO: 36-FR2 of the heavy chain variable domain of scFv 11
MSWIRQAPGKGLEWVSY

| SEQ ID NO | Description |
|---|---|
| 46 | scFv 13 |
| 47 | scFv 14 |
| 48 | scFv 15 |
| 49 | Karunyam 13 |
| 50 | Karunyam 14 |
| 51 | Karunyam 15 |
| 52 | FR1 of the heavy chain variable domain of scFv 13 |
| 53 | CDR1 of the heavy chain variable domain of scFv 13 |
| 54 | FR2 of the heavy chain variable domain of scFv 13 |
| 55 | CDR2 of the heavy chain variable domain of scFv 13 |
| 56 | FR3 of the heavy chain variable domain of scFv 13 |
| 57 | FR1 of the light chain variable domain of scFv 13 |
| 58 | CDR1 of the light chain variable domain of scFv 13 |
| 59 | FR2 of the light chain variable domain of scFv 13 |
| 60 | CDR2 of the light chain variable domain of scFv 13 |
| 61 | FR3 of the light chain variable domain of scFv 13 |
| 62 | FR1 of the heavy chain variable domain of scFv 14 |
| 63 | CDR1 of the heavy chain variable domain of scFv 14 |
| 64 | FR2 of the heavy chain variable domain of scFv 14 |

SEQ ID NO: 37-CDR2 of the heavy chain variable domain of scFv 11
ISSSGSTI
SEQ ID NO: 38-FR3 of the heavy chain variable domain of scFv 11
YYADSVKGRFTISRDNAKNSLYLQMNSLRAEDTAVYYC
SEQ ID NO: 39-CDR3 of the heavy chain variable domain of scFv 11
ARDFSHQQLVPSWGQGTLVTV
SEQ ID NO: 40-FR1 of the light chain variable domain of scFv 11
DIQLTQSPSFLSASVGDRVTITCRAS
SEQ ID NO: 41-CDR1 of the light chain variable domain of scFv 11
QGISSY
SEQ ID NO: 42-FR2 of the light chain variable domain of scFv 11
LAWYQQKPGKAPKLLIY
SEQ ID NO: 43-CDR2 of the light chain variable domain of scFv 11
AAS
SEQ ID NO: 44-FR3 of the light chain variable domain of scFv 11
TLQSGVPSRFSGSGSGTEFTLTISSLOPEDFATYYC
SEQ ID NO: 45-CDR3 of the light chain variable domain of scFv 11
QQLNSYPLTFGGGTKVEIKVD

| | |
|---|---|
| 65 | CDR2 of the heavy chain variable domain of scFv 14 |
| 66 | FR3 of the heavy chain variable domain of scFv 14 |
| 67 | FR1 of the light chain variable domain of scFv 14 |
| 68 | CDR1 of the light chain variable domain of scFv 14 |
| 69 | FR2 of the light chain variable domain of scFv 14 |
| 70 | CDR2 of the light chain variable domain of scFv 14 |
| 71 | FR3 of the light chain variable domain of scFv 14 |
| 72 | FR1 of the heavy chain variable domain of scFv 15 |
| 73 | CDR1 of the heavy chain variable domain of scFv 15 |
| 74 | FR2 of the heavy chain variable domain of scFv 15 |
| 75 | CDR2 of the heavy chain variable domain of scFv 15 |
| 76 | FR3 of the heavy chain variable domain of scFv 15 |
| 77 | FR1 of the light chain variable domain of scFv 15 |
| 78 | CDR1 of the light chain variable domain of scFv 15 |
| 79 | FR2 of the light chain variable domain of scFv 15 |
| 80 | CDR2 of the light chain variable domain of scFv 15 |
| 81 | FR3 of the light chain variable domain of scFv 15 |
| 82 | scFv 18 |
| 83 | scFv 19 |
| 84 | scFv 20 |
| 85 | Karunyam 18 |
| 86 | Karunyam 19 |
| 87 | Karunyam 20 |
| 88 | FR1 of the heavy chain variable domain of scFv 18 |
| 89 | CDR1 of the heavy chain variable domain of scFv 18 |
| 90 | FR2 of the heavy chain variable domain of scFv 18 |
| 91 | CDR2 of the heavy chain variable domain of scFv 18 |
| 92 | FR3 of the heavy chain variable domain of scFv 18 |
| 93 | CDR3 of the heavy chain variable domain of scFv 18 |
| 94 | FR1 of the light chain variable domain of scFv 18 |
| 95 | CDR1 of the light chain variable domain of scFv 18 |
| 96 | FR2 of the light chain variable domain of scFv 18 |
| 97 | CDR2 of the light chain variable domain of scFv 18 |
| 98 | FR3 of the light chain variable domain of scFv 18 |
| 99 | CDR3 of the light chain variable domain of scFv 18 |
| 100 | FR1 of the heavy chain variable domain of scFv 19 |
| 101 | CDR1 of the heavy chain variable domain of scFv 19 |
| 102 | FR2 of the heavy chain variable domain of scFv 19 |
| 103 | CDR2 of the heavy chain variable domain of scFv 19 |
| 104 | FR3 of the heavy chain variable domain of scFv 19 |
| 105 | CDR3 of the heavy chain variable domain of scFv 19 |
| 106 | FR1 of the light chain variable domain of scFv 19 |
| 107 | CDR1 of the light chain variable domain of scFv 19 |
| 108 | FR2 of the light chain variable domain of scFv 19 |
| 109 | CDR2 of the light chain variable domain of scFv 19 |
| 110 | FR3 of the light chain variable domain of scFv 19 |
| 111 | CDR3 of the light chain variable domain of scFv 19 |
| 112 | FR1 of the heavy chain variable domain of scFv 20 |
| 113 | CDR1 of the heavy chain variable domain of scFv 20 |
| 114 | FR2 of the heavy chain variable domain of scFv 20 |
| 115 | CDR2 of the heavy chain variable domain of scFv 20 |
| 116 | FR3 of the heavy chain variable domain of scFv 20 |
| 117 | CDR3 of the heavy chain variable domain of scFv 20 |
| 118 | FR1 of the light chain variable domain of scFv 20 |
| 119 | CDR1 of the light chain variable domain of scFv 20 |
| 120 | FR2 of the light chain variable domain of scFv 20 |
| 121 | CDR2 of the light chain variable domain of scFv 20 |
| 122 | FR3 of the light chain variable domain of scFv 20 |
| 123 | CDR3 of the light chain variable domain of scFv 20 |
| 124 | Chitin-binding domain |
| 125 | Tachystatin A2 Tag |
| 126 | Karunyam 21 |
| 127 | Karunyam 22 |
| 128 | Karunyam 23 |
| 129 | Karunyam 24 |
| 130 | ACE2 receptor |
| 131 | ACE2 receptor mutant M1 |
| 132 | ACE2 receptor mutant M2.1 |
| 133 | ACE2 receptor mutant M2.2 |
| 134 | ACE2 receptor mutant M3 |
| 135 | ACE2 receptor mutant M4 |
| 136 | Karunyam 25 |
| 137 | Karunyam 26 |
| 138 | Karunyam 27 |
| 139 | Karunyam 28 |
| 140 | Karunyam 29 |
| 141 | Karunyam 30 |

1. An anti-virus moiety immobilised in a matrix, wherein the matrix is suitable for use as a hygiene product.

2. The anti-virus moiety of claim 1, wherein the anti-virus moiety is an antibody.

3. The anti-virus moiety of claim 1, wherein the anti-virus moiety specifically binds to an epitope in a coronavirus with a K_{D} value of between 1 to 10 nM.

4. The anti-virus moiety of claim 1, wherein the anti-virus moiety comprises an amino acid sequence having ≥70% sequence identity with any one of SEQ ID NOs: 1 to 3, 46 to 48 and 82 to 84.

5. The anti-virus moiety of claim 1, wherein the anti-virus moiety is immobilised in the matrix in a reversible manner.

6. The anti-virus moiety of claim 1, wherein the matrix is cellulose, starch, collagen, insect shells, mucous membrane, or an inorganic matrix.

7. The anti-virus moiety of claim 1, wherein the anti-virus moiety is coupled to a matrix-binding domain.

8. The anti-virus moiety of claim 7, wherein:
(a) the matrix is cellulose and the matrix-binding domain comprises SEQ ID NO: 6.
(b) the matrix is chitosan and the matrix-binding domain is a chitin binding domain, e.g. comprises SEQ ID NO: 124; or
(c) the matrix is polyester and the matrix-binding domain is a tachystatin affinity tag, e.g. comprises SEQ ID NO: 125.

9. The anti-virus moiety of claim 1, wherein the matrix is part of the hygiene product.

10. (canceled)

11. (canceled)

12. A hygiene product comprising the anti-virus moiety of claim 1.

13. The anti-virus moiety of claim 1, wherein the hygiene product is:
(a) a personal protective equipment, lab coat, protective mask, (e.g. face mask, mask, respirator, face shield, surgical mask, medical mask, filter mask, mouth mask, gas mask, shoe cover, hair cap, veil or hijab;
(b) a filter, an air filter, HEPA filter or a water filter;
(c) a polyethylene membrane;
(d) a household product in the form of a sheet, tissue, surface wipe, linen, napkin, curtain, or table cloth;
(e) a household product in the form of a liquid or semi-liquid, cleaning solution, aerosol spray, sanitiser gel or cream; or
(f) a cellulose solution.

14. A method of filtering or removing virus particles and/or fragments thereof from an environment or a surface, comprising applying the anti-virus moiety of claim 1 to filter or remove virus particles and/or fragments thereof from the environment or a surface.

15. (canceled)

16. The method of claim 14, wherein the method is for preventing virus transmission.

17. The method of claim 14, wherein the virus is SARS-COV-2.

18. The anti-virus moiety of claim 2, wherein the antibody is a single chain variable fragment.

19. The anti-virus moiety of claim 1, wherein the anti-virus moiety specifically binds to an epitope in SARS-COV-2.

20. The anti-virus moiety of claim 1, wherein the anti-virus moiety is comprised in the matrix.

21. The anti-virus moiety of claim 20, wherein the matrix is cellulose, starch, collagen, insect shells, mucous membranes, or an inorganic matrix.

22. The hygiene product of claim 12, wherein the hygiene product is:
(a) a personal protective equipment, lab coat, protective mask, face mask, mask, respirator, face shield, surgical mask, medical mask, filter mask, mouth mask, gas mask, shoe cover, hair cap, veil or hijab;
(b) a filter, an air filter, HEPA filter or a water filter;
(c) a polyethylene membrane;
(d) a household product in the form of a sheet, tissue, surface wipe, linen, napkin, curtain, or table cloth;
(e) a household product in the form of a liquid or semi-liquid, cleaning solution, aerosol spray, sanitiser gel or cream; or
(f) a cellulose solution.

23. The anti-virus moiety of claim 1, wherein the anti-virus moiety is comprised in a fusion protein comprising a matrix-binding domain.
